(19) United States
(12) Patent Application Publication
(10) Pub. No.: US 2020/0305739 A1
(43) Pub. Date: Oct. 1, 2020

(54) PORTABLE SCANNING DEVICE AND PROCESSING SYSTEM

(71) Applicant: Anya L. Getman, Sandy, OR (US)

(72) Inventor: Anya L. Getman, Sandy, OR (US)

(21) Appl. No.: 16/836,704

(22) Filed: Mar. 31, 2020

Related U.S. Application Data
(60) Provisional application No. 62/827,193, filed on Apr. 1, 2019.
(60) Provisional application No. 62/827,195, filed on Apr. 1, 2019.

(51) Int. Cl. A61B 5/024; A61B 5/04

(52) U.S. Cl. CPC A61B 5/02438; A61B 5/04

(57) ABSTRACT

A portable device for diagnosing medical conditions and/or physiological reactions includes internal sensors, cameras, mirrors, lasers, electronics, speakers and/or other components. Sensors may be mounted on one or more internal gimbals for stability. The device transmits data it obtains to be processed. Deep learning and other algorithms and tools may be employed to make a diagnosis, detect physiological reactions, or make other observations from data received from the device, as desired. Information from the analysis may be communicated to the device for display. In one embodiment, the device is in the shape of a common item such as a coffee cup. In another embodiment, the device is in the form of a musical instrument and is configured to sense a subject's reaction to music, beats, and/or other stimuli.

### RELATED APPLICATIONS

This application is a non-provisional application of U.S. Provisional Application No. 62/827,193, which was filed on Apr. 1, 2019, the contents of which are herein incorporated by reference in their entirety for all purposes; and this application is a non-provisional application of U.S. Provisional Application No. 62/827,195, which was filed on Apr. 1, 2019, the contents of which are herein incorporated by reference in their entirety for all purposes.

### TECHNICAL FIELD

The field of this disclosure relates generally to portable medical assessment devices and, in particular, to a portable assessment device that can be used for medical diagnosis and optionally also as an actuator (dual use for the active sensors) to treat certain conditions.

### BACKGROUND INFORMATION

In industry, machine vision provides imaging-based automatic inspection and analysis in automatic inspection, process control, robot guidance, and other applications. While systems vary, machine vision can incorporate software and hardware products, integrated systems, actions, methods and expertise.

In one machine vision configuration, one or more cameras acquire an image. The image is then processed. A CPU, a GPU, a FPGA or a combination of these can perform the processing. Deep learning training and inference impose higher processing performance requirements. Multiple stages of processing are generally used in a sequence that ends up as a desired result. A typical sequence might start with tools such as filters which modify the image, followed by extraction of objects, then extraction of data from those objects. The data can be communicated and/or compared against target values to create and communicate “pass/fail” results.

Some machine vision image processing methods include: stitching, filtering, thresholding, pixel counting, segmentation, edge detection, color analysis, blob detection and extraction, neural net/deep learning/machine learning, pattern recognition, comparison against target values, as well as others.

While machine vision has been implemented in industry, there is an unmet need in the life sciences, for example, for detecting, processing, learning from, and displaying data about aspects of animals and human beings. This includes medical and psychological diagnosis, as well as understanding human reaction to music and other stimuli.

### OVERVIEW OF DISCLOSURE

In one embodiment, a portable detection system that detects for physiological conditions in an animal body comprises: multiple radiation emitters for emitting multiple forms of radiation toward an animal body; multiple sensors for obtaining, in response to the multiple forms of radiation, body data representative of an area of the animal body; and a communication nexus that operatively connects the emitters and sensors to a processing tool for processing the body data to identify the presence of one or more components or conditions of the animal body, wherein the processing tool comprises a trainable artificial intelligence system (AI system).

In some additional, alternative, or selectively cumulative embodiments, a method for identifying a physiological condition in an animal body comprises: employing emitting multiple forms of radiation toward an animal body; obtaining, in response to the multiple forms of radiation, body data representative of an area of the animal body; providing the body data to a processing tool to identify the presence of one or more components or conditions of the animal body, wherein the processing tool comprises a trainable AI system; and providing information concerning the component or condition.

In some additional, alternative, or selectively cumulative embodiments, a physiological detection system comprises: a scanner for monitoring a body of an animal, the scanner producing difference images of a component or condition of the body between an initial state and a post-stimulus state; processing software and/or hardware for processing the difference images to determine whether the component or condition of the body has changed to an altered state in which a difference is identified between the initial state and the post-stimulus state in response to a stimulus, the processing software and/or hardware including a trainable artificial intelligence (AI) system to learn the difference between the initial state and the altered state; and an adjustable stimulus source that is optionally directable toward the body or toward a component or condition of the body to create the post-stimulus state.

In some additional, alternative, or selectively cumulative embodiments, a detection system for detecting physiological reactions in an animal body comprises: image signal means for producing an image signal representative of an image of an area of the animal body; and processing means for processing the image signal to identify the presence of one or more components or conditions of the animal body, wherein the means for processing includes: an optional digitizing means for producing a digitized image signal whenever the image signal is not already digitized; an optional detection zone means for specifying a detection zone surrounding the one or more components or conditions of the animal body within the image and means for extracting a portion of the digitized image signal corresponding to the detection zone to produce a digitized detection zone signal; tiling means for producing a tiled detection zone pixel map from the digitized detection zone signal; a trainable artificial intelligence system (AI system) comprising input processing units, hidden processing units and output processing units wherein an output from each of the input processing units is connected to an input of each of the hidden processing units and an output from each of the hidden processing units is connected to an input of each of the output processing units and wherein the trainable AI system produces an output signal at each of the output processing units representative of one or more characteristics of one or more of the components or conditions within the portion of the detection zone; inputting means for inputting the tiled detection zone pixel map into the trainable AI system; an adjustable stimulus source that is optionally directable toward the area or the detection zone of the animal body; and an output filter for producing a presence output signal indicating that one or more characteristics of the one or more components or conditions of the animal body within the detection zone has changed by a significant amount in a predetermined manner in response to the stimulus, by performing a matched filter operation on a time series of the detection zone pixel maps, including at least one detection zone pixel map for a time period before the stimulus and at least one detection zone pixel map for a time period after the stimulus to detect whether one or more characteristics of the one or more components or conditions of the animal body has changed in response to the stimulus.

In some additional, alternative, or selectively cumulative embodiments, a method for identifying a physiological response in response to a stimulus comprises: producing an image signal representative of an image of an area of the animal body; processing the image signal to identify the presence of one or more components or conditions of the animal body, optionally producing a digitized image signal whenever the image signal is not already digitized; specifying a detection zone surrounding the one or more components or conditions of the animal body within the image and means for extracting a portion of the digitized image signal corresponding to the detection zone to produce a digitized detection zone signal; producing a tiled detection zone pixel map from the digitized detection zone signal; inputting the tiled detection zone pixel map into a trainable artificial intelligence system (AI system) comprising input processing units, hidden processing units and output processing units wherein an output from each of the input processing units is connected to an input of each of the hidden processing units and an output from each of the hidden processing units is connected to an input of each of the output processing units and wherein the trainable AI system produces an output signal at each of the output processing units representative of one or more characteristics of one or more of the components or conditions within the portion of the detection zone; providing an adjustable stimulus that is optionally directable toward the area or the detection zone of the animal body; and employing an output filter for producing a presence output signal indicating that one or more characteristics of the one or more components or conditions of the animal body within the detection zone has changed by a significant amount in a predetermined manner in response to the stimulus, by performing a matched filter operation on a time series of the detection zone pixel maps, including at least one detection zone pixel map for a time period before the stimulus and at least one detection zone pixel map for a time period after the stimulus to detect whether one or more characteristics of the one or more components or conditions of the animal body has changed in response to the stimulus.

In some additional, alternative, or selectively cumulative embodiments, a method for scanning the body of an animal to determine whether a component or condition of the body has changed between an initial state and a post-stimulus state in response to a stimulus comprises: scanning the body of the animal to collect initial images of the component or condition of the body in the initial state; providing a stimulus to the body; scanning the body of the animal to collect post-stimulus images of the component or condition of the body in the post-stimulus state; and employing a trainable AI system, which is trained to essentially correctly identify differences between multiple images of the component or difference between multiple images of the condition of the body, to determine significant differences between the initial images of the initial state and the post-stimulus state to determine whether the post-stimulus state is an altered state.

In some additional, alternative, or selectively cumulative embodiments, a system including a portable device with a detection system for detecting medical conditions or physiological reactions of a subject (such as a human) comprises a housing shaped to disguise the device as an everyday object, such as a coffee mug or musical instrument; a plurality of sensors within the device, at least one of the sensors being a camera; a wall of the device being made of a material that is opaque or reflective when viewed from the exterior but is translucent or transparent when viewed from the interior of the device looking out toward the exterior; the device being in communication with a trainable artificial intelligence system that analyses data about a subject that the sensors gather, to identify a medical condition or physiological reaction in the subject; the artificial intelligence system being adapted to transmit data relating to the medical condition or physiological reaction to the device, the device having a display screen to display data from the artificial intelligence system.

In some additional, alternative, or selectively cumulative embodiments, a system including a portable device with a detection system for detecting medical conditions or physiological reactions of a subject comprises: a housing shaped to disguise the device as an everyday object; a plurality of sensors within the housing and arranged in an array that is moveable within the housing; the system including a trainable artificial intelligence system that analyses data about a subject that the sensors gather, to identify a medical condition or physiological reaction in the subject, the artificial intelligence system configured to utilize a deep learning neural network and an image and anomaly database; the device including a display to display data from the artificial intelligence system.

In some additional, alternative, or selectively cumulative embodiments, a system including a portable device with a detection system for detecting physiological or emotional reactions of a subject comprises: a housing shaped as a musical instrument; a plurality of sensors within the housing; an audio speaker with a volume control; the system including a trainable artificial intelligence system that analyses data about a subject that the sensors gather, to identify a physiological or emotional response in the subject in reaction to sounds that the device plays; and a display affiliated with the device to display data from the artificial intelligence system.

In some additional, alternative, or selectively cumulative embodiments, an altered state is evaluated to determine existence of a change in a psychological state or emotional state.

In some additional, alternative, or selectively cumulative embodiments, the stimulus is modified to enhance or diminish the change in a psychological state or emotional state.

In some additional, alternative, or selectively cumulative embodiments, the artificial intelligence (AI) comprises one or more of a neural network, a probabilistic technique such as Bayes or Markov algorithm, a kernel method (like SVM, decision trees/random forest, Gaussians, PCA, can-cor . . . ), reinforcement learning that can have nothing to do with artificial neural networks, artificial reasoning a.k.a. “good old fashioned AI,” many path-planning and intelligent control-systems methods that correspond to “classical AI” (not the same as GOFAI), alife (swarms, cellular automata . . . ), agents and chaos systems, and/or any algorithm or group of algorithms that optimize a value function (reinforcement learning and linear dynamic programming).

In some additional, alternative, or selectively cumulative embodiments, the trainable AI system is comprised of a single layer of input processing units, more than one layer of hidden processing units and a single layer of output processing units and wherein an output from each of the input processing units is connected to an input of each of the hidden processing units in a first layer of the hidden processing units, an output from each of the hidden processing units in a last layer of the hidden processing units is connected to an input of each of the output processing units, and multiple layers of the hidden processing units are interconnected such that the output from each of the hidden processing units in any one but the last of the layers of the hidden processing units is connected to the input of each of the hidden processing units in a next layer of the hidden processing units.

In some additional, alternative, or selectively cumulative embodiments, the animal body is a human body.

In some additional, alternative, or selectively cumulative embodiments, one of the one or more components or conditions of the animal body comprises an internal component.

In some additional, alternative, or selectively cumulative embodiments, one of the one or more components of the animal body comprises one or more of an internal organ or an internal system.

In some additional, alternative, or selectively cumulative embodiments, one of the one or more components of the animal body comprises one or more of a blood vessel or a nerve.

In some additional, alternative, or selectively cumulative embodiments, one of the one or more conditions of the animal body comprises one or more of heart rate, blood pressure, pupil diameter.

In some additional, alternative, or selectively cumulative embodiments, one of the one or more conditions of the animal body comprises an emotional condition.

In some additional, alternative, or selectively cumulative embodiments, one of the one or more conditions of the animal body comprises one or more of sorrow, joy, or arousal.

In some additional, alternative, or selectively cumulative embodiments, the representative body data comprises an image.

In some additional, alternative, or selectively cumulative embodiments, the representative body data comprises an infrared image.

In some additional, alternative, or selectively cumulative embodiments, the representative body data comprises a sound image.

In some additional, alternative, or selectively cumulative embodiments, the representative body data comprises an ultrasonic sound image.

In some additional, alternative, or selectively cumulative embodiments, the sensors include one or more sensors for producing a video signal representative of a video image of the one or more components or conditions of the human body.

In some additional, alternative, or selectively cumulative embodiments, the sensors include a video camera for producing an analog video signal representative of a video image.

In some additional, alternative, or selectively cumulative embodiments, the sensors comprise an infrared sensor.

In some additional, alternative, or selectively cumulative embodiments, the sensors comprise a sound sensor.

In some additional, alternative, or selectively cumulative embodiments, the sensors comprise an ultrasound sensor.

In some additional, alternative, or selectively cumulative embodiments, a stimulus is directed toward the body of the animal.

In some additional, alternative, or selectively cumulative embodiments, the stimulus is visual.

In some additional, alternative, or selectively cumulative embodiments, the stimulus is auditory.

In some additional, alternative, or selectively cumulative embodiments, in the stimulus comprises sound.

In some additional, alternative, or selectively cumulative embodiments, herein the stimulus comprises music.

In some additional, alternative, or selectively cumulative embodiments, the stimulus comprises one or more selected harmonies.

In some additional, alternative, or selectively cumulative embodiments, the stimulus comprises one or more selected chords.

In some additional, alternative, or selectively cumulative embodiments, the stimulus comprises ultrasound.

In some additional, alternative, or selectively cumulative embodiments, one or more of the radiation emitters can be applied to treat the component or the condition.

In some additional, alternative, or selectively cumulative embodiments, the component or the condition comprises Alzheimer's disease.

In some additional, alternative, or selectively cumulative embodiments, the component or the condition comprises a fibroid.

In some additional, alternative, or selectively cumulative embodiments, the radiation comprises one or more of UV light radiation, visible light radiation, infrared light radiation, microwave radiation, radio sound radiation, and ultrasonic radiation.

In some additional, alternative, or selectively cumulative embodiments, the trainable AI system is implemented in computer software as a neural network simulator running on a computer.

In some additional, alternative, or selectively cumulative embodiments, the trainable AI system is implemented in computer hardware.

In some additional, alternative, or selectively cumulative embodiments, a numerical connection weight is assigned to (i) each of the connections between each of the outputs of each of the input processing units and each of the inputs of each of the hidden processing units and (ii) each of the connections between each of the outputs of each of the hidden processing units and each of the inputs of each of the output processing units.

In some additional, alternative, or selectively cumulative embodiments, a numerical bias is assigned to each of the hidden processing units and each of the output processing units.

In some additional, alternative, or selectively cumulative embodiments, the value of each of the numerical connection weights and each of the numerical biases is determined through a closed-loop training procedure utilizing backpropagation techniques.

In some additional, alternative, or selectively cumulative embodiments, the closed-loop training procedure is the Generalized Delta Rule.

In some additional, alternative, or selectively cumulative embodiments, the stimulus causes the characteristic to respond in a desirable manner.

In some additional, alternative, or selectively cumulative embodiments, the change in the characteristic can be identified.

In some additional, alternative, or selectively cumulative embodiments, the change in the characteristic can be positively reinforced by a subsequent stimulus.

In some additional, alternative, or selectively cumulative embodiments, a subsequent stimulus can be modified to affect the change in the characteristic.

In some additional, alternative, or selectively cumulative embodiments, a subsequent stimulus can be modified to enhance the change in the characteristic.

In some additional, alternative, or selectively cumulative embodiments, the processing means includes a computer simulating the neural network.

In some additional, alternative, or selectively cumulative embodiments, a presence output or presence signal is triggered by a processing tool or a computer when an altered state is determined to exist in the component or condition.

In some additional, alternative, or selectively cumulative embodiments, the image of the area is comprised of pixels, each pixel having a value corresponding to the amount of light associated with the pixel; and wherein the computer compares the values of pixels of a most recent image of the area with the values of pixels of an earlier in time image of the area to produce a difference image comprised of pixels, each of which have a value corresponding to the difference in values between corresponding pixels of the most recent image and the earlier in time image; and wherein the AI system or neural network simulated by the computer or processing means having weights for each pixel network simulated by the computer having weights for each pixel which are multiplied by the respective pixel value of the difference image and then added together to form a sum, which if greater than a predetermined amount, results in the computer or processing means providing the presence output or presence signal.

In some additional, alternative, or selectively cumulative embodiments, the scanning system or monitoring means includes additional sensors, that together with the video camera produces the difference image of the area, the value of each of the pixels of the image having a component corresponding to the additional sensors as well as a component corresponding to the amount of light associated with the pixel.

In some additional, alternative, or selectively cumulative embodiments, additional sensors include at least a second video camera.

In some additional, alternative, or selectively cumulative embodiments, additional sensors include infrared detectors.

In some additional, alternative, or selectively cumulative embodiments, additional sensors include microwave detectors.

In some additional, alternative, or selectively cumulative embodiments, the trainable neural network uses back propagation techniques.

In some additional, alternative, or selectively cumulative embodiments, the detection system is portable.

In some additional, alternative, or selectively cumulative embodiments, the detection system is handheld.

In some additional, alternative, or selectively cumulative embodiments, the detection system is housed in a mug-shaped container.

In some additional, alternative, or selectively cumulative embodiments, where the scans are directed at an object instead of a body.

Some additional, alternative, or selectively cumulative embodiment, of the present invention relate to a system that includes a portable device that has a detection system for detecting medical conditions or physiological reactions of a human. The device has a housing shaped to disguise the device as an everyday object, such as a coffee mug or musical instrument. A plurality of sensors is located within the device, with at least one of the sensors being a camera. A wall of the device is made of a material that is opaque or reflective when viewed from the exterior, but the wall is translucent or transparent when viewed from the interior of the device looking out toward the exterior. In this way, light from outside the device can be detected by the sensors on the interior of the device, while the subject does not see the sensors on the interior of the device.

The device is in communication with a trainable artificial intelligence system that analyses data about a subject that the sensors gather. The system may identify a medical condition or physiological reaction in the subject. After processing data from the sensors, the artificial intelligence system is adapted to transmit data relating to the medical condition or physiological reaction to the device, the device having a display screen to display data from the artificial intelligence system.

Various optional features may be incorporated, either alone or in combination with other optional features, into the system. At least one sensor within the device may mounted on a gimbal to stabilize the sensor. The gimbal may rotate about one, two, or three axes, as desired. The device may include a variety of configuration, such as a first camera mounted on a gimbal, and a second camera mounted on a different, rotatable mounting.

In some additional, alternative, or selectively cumulative embodiments, the device has multiple separate housings, such as two halves. As just one example, a first half may be mounted on a table top, while the second half may be mounted on a bottom side of the table top. The first and second halves are typically in communication with one another. In one embodiment, the device is shaped as a coffee cup. The cup may have a wall that is reflective when viewed from the exterior and at least partially transparent viewed from the interior of the cup.

In some additional, alternative, or selectively cumulative embodiments, the device includes an array of cameras and/or sensors arranged in a spiral configuration within the device. As an option, a laser or other light source may be located on top of the array. In another embodiment, the device may include active sensors employed in sender/receiver pairs. In one configuration, the sensors may be mounted on a pole that a drive motor rotates. The drive motor may be operated from a control panel on the device, or alternatively remotely as from a cell phone, laptop, or other external device.

In some additional, alternative, or selectively cumulative embodiments, sensors are provided in a detachable unit that is attached onto the cup. Consequently, a portion of the cup may be available to hold a beverage for drinking and/or another purpose.

In some additional, alternative, or selectively cumulative embodiments, the device may be equipped with sensors to determine the orientation, location, velocity, acceleration, and/or other aspects of the device. In one embodiment, the device includes a gyro and an accelerometer to determine one or more orientation parameters of the cup.

In some additional, alternative, or selectively cumulative embodiments, the device may be shaped as a musical instrument. The instrument may be sound-emitting, as through a speaker that is either part of the device or is external to the device, including a BlueTooth-connected speaker, headphones, earbuds or other device that emits sound from an electronic signal. In one embodiment, the sensors gather data that may be processed to sense a physiological and/or emotional response of the subject to emitted sounds.

In some additional, alternative, or selectively cumulative embodiments, a trainable artificial intelligence system includes a deep learning neural network, an image and anomaly database, and a customer medical and image history record. Alternatively, the deep learning system may access one or more other databases or sources of information as it seeks to identify potential medical conditions and/or physiological or emotional responses of a subject.

In some additional, alternative, or selectively cumulative embodiments, the medical condition or the physiological reaction is associated with an internal organ, an internal system, a blood vessel, or a nerve, heart rate, blood pressure, pupil diameter, an emotional condition, a facial expression, tearing up, swaying, or change in position.

Some additional, alternative, or selectively cumulative embodiments of the present invention relate to a system including a portable device with a detection system for detecting medical conditions or physiological reactions of a human. The device may include a housing shaped to disguise the device as an everyday object. A plurality of sensors resides within the housing and at least some of the sensors are arranged in an array that is moveable within the housing. The system may include a trainable artificial intelligence system that analyses data about a subject that the sensors gather, to identify a medical condition or physiological reaction in the subject, the artificial intelligence system including a deep learning neural network and an image and anomaly database, and/or other databases or information useful in analyzing data from the sensors. The device may include a display to display data from the artificial intelligence system. In one embodiment, the device is shaped as a coffee mug and the screen is circular in configuration and resides in the top opening of the device.

Devices according to this embodiment may include optional features as described above. Further, the device may include other optional features, either alone or in combination with one another. One embodiment includes a device that has a motor on the interior of the device to selectively move the sensors within the housing. In another embodiment, the device includes active sensors employed in sender/receiver pairs, the sensors mounted on a pole that a drive motor rotates. The device may optionally include at least one sensor within the interior of the device is moveable by remote command.

In some additional, alternative, or selectively cumulative embodiments, the system includes multiple portable devices each having sensors on the interior thereof. The multiple devices are in communication with one another and, for example, may transmit information such as the location of the device, data from sensors, movement characteristics of the device, and other information.

In some additional, alternative, or selectively cumulative embodiments, a system according to the present invention may include a portable device with a detection system for detecting physiological and/or emotional reactions of a human. The device may have a housing shaped as a musical instrument, which may be molded, 3D printed, or constructed by other means. A plurality of sensors resides within the housing. The system also includes at least one audio speaker that has a volume control. The system includes a trainable artificial intelligence system that analyses data about a subject that the sensors gather, to identify a physiological or emotional response in the subject in reaction to sounds that the device plays. The device includes a display affiliated with the device to display data from the artificial intelligence system.

In some additional, alternative, or selectively cumulative embodiments, the sounds are a series of at least one of music, binaural beats, or a series of tones, among other possible series of sounds. In one embodiment, the sensors detect at least one of: heart rate, blood pressure, pupil diameter, facial expression, tearing up, swaying to music, change in seating position. The artificial intelligence system is adapted to identify psychological or emotional states in the subject in response to musical stimuli.

In some additional, alternative, or selectively cumulative embodiments, at least one sensor within the device is mounted on a gimbal to stabilize the sensor.

In some additional, alternative, or selectively cumulative embodiments, the device includes a first camera mounted on a gimbal and a second camera mounted so as to be rotatable.

In some additional, alternative, or selectively cumulative embodiments, the device comprises two halves, including a first half adapted to be mounted on a top side of a table top and a second half adapted to be mounted on a bottom side of the table top, the first and second halves being in communication with one another.

In some additional, alternative, or selectively cumulative embodiments, the device is shaped as a cup, having a cup wall that is reflective when viewed from the exterior and at least partially transparent when viewed from the interior of the cup to the exterior.

In some additional, alternative, or selectively cumulative embodiments, the device includes an array of cameras and/or sensors arranged in a spiral configuration within the device.

In some additional, alternative, or selectively cumulative embodiments, the laser is located on top of the array.

In some additional, alternative, or selectively cumulative embodiments, the device includes active sensors employed in sender/receiver pairs, the sensors mounted on a pole that a drive motor rotates.

In some additional, alternative, or selectively cumulative embodiments, the sensors are provided in a detachable unit that is attached onto the device, a portion of the device adapted to hold a beverage, wherein the unit includes a rechargeable battery.

In some additional, alternative, or selectively cumulative embodiments, the device includes a gyro and an accelerometer to determine one or more orientation parameters of the device.

In some additional, alternative, or selectively cumulative embodiments, the device is shaped as a musical instrument, is operable to emit sounds, and is operable to sense a physiological and/or emotional response of the subject to sounds emitted by the device.

In some additional, alternative, or selectively cumulative embodiments, the trainable artificial intelligence system utilizes a deep learning neural network, an image and anomaly database, and a medical and image history record of the subject.

Selectively cumulative embodiments are embodiments that include any combination of multiple embodiments that are not mutually exclusive.

The foregoing and other objects, features, and advantages of the invention will become more apparent from the following detailed description, which proceeds with reference to the accompanying figures.

### BRIEF DESCRIPTION OF THE DRAWINGS

FIG. 1 illustrates an example of a portable assessment device (such as shaped like one or more mugs) used as a scanner to assess medical or threat conditions of a user.

FIG. 2 illustrates the mechanism inside the mugs of FIG. 1.

FIGS. 3A-3C further illustrate the mechanism inside the mugs of FIG. 1.

FIGS. 4A and 4B illustrate an array of sensors inside the mug.

FIGS. 5A and 5B illustrate an alternative array of sensors.

FIGS. 6A and 6B illustrates another alternative array of sensors.

FIG. 7 is a process diagram showing analytical steps that may be associated with assessment of the condition of the user.

FIG. 8 shows examples of sensor inputs and outputs associated with a portable assessment device.

FIG. 9 shows examples of how a portable assessment device can sense and adjust to environmental conditions.

FIGS. 10A-10D illustrate a four-step process for analyzing images.

FIGS. 11A-11D illustrate a step in visually analyzing a blueberry muffin.

FIGS. 12A-12D illustrate another step in analyzing an image of a blueberry muffin.

FIGS. 13A-13D illustrate a step in analyzing the face of a small dog.

FIGS. 14A-14D illustrate an image of multiple small dogs for analysis.

FIG. 15 illustrates a sensor device shaped as a violin, which senses aspects of a person in front of the sensor device.

### DETAILED DESCRIPTION OF EMBODIMENTS

Example non-limiting embodiments are described below with reference to the accompanying drawings.

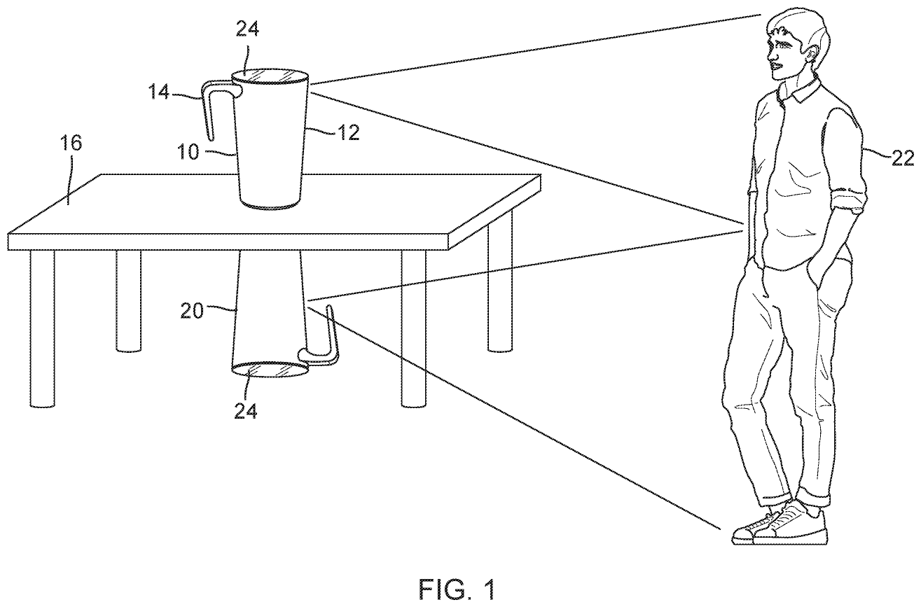

Turning now to one non-limiting specific implementation of the invention, FIG. 1 illustrates a device 10 that may have the form and look disguises the device so that it is not readily apparent that it is a detection system. More particular, the device may have a shape appearance that looks different from a tricorder or other medical scanning device. In some embodiments, the device 10 may have the appearance of an everyday object.

An everyday object may be a household object such as an appliance, piece of equipment, drinkware, furniture, artwork, or toy, etc. Examples of an appliance include, but are not limited to, a microwave, a coffee maker, and a drink dispenser. Examples of a piece of equipment include, but are not limited to, a phone, a printer, a laptop computer, a monitor, or a speaker, etc. Examples of drinkware include, but are not limited to, a cup, a mug, a glass, or a coffee cup, etc. Examples, of furniture include, but are not limited to, a chair, a sofa, a table, a desk, or a cabinet, etc. Examples of artwork include, but are not limited to, a wall picture or a statute, etc. Examples, of toys include, but are not limited to, a robot or a stuffed animal, etc. The device 10 may act as a single device 10 or may interact with multiple devices in the same room or different room that have the same appearance or functions or have different appearances or functions. A primary device 10 may be portable. In particular, a primary device 10 may be handheld.

The interior of the device 10 may include sensors, mirrors, electronics, speakers, beepers and the like. The sensors may include one or more of scanning sensors or self-relational sensors. Scanning sensors may include, but are not limited to, image sensors (like a camera), auditory sensors (like a microphone), ultrasonic sensors, infrared sensors, etc. In some embodiments that include two or more sensors of the same category, the sensors may be identical or have different makes or ranges of operation. Image sensors may, for example, have different optical arrangements such as different focal arrangements and fields of view. Self-relational sensors may include, but are not limited to, accelerometers, gyroscopes, and GPS. The sensors may be packaged so that they are modular, so that they can be interchangeably connected to the device 10. One will appreciate that certain sensors may perform better in particular locations or orientations. In such circumstances, particular categories of sensors may be shaped differently to accommodate particular positioning.

The device may also include one or more stimuli emitters. The stimuli emitters may include radiation emitters including, but not limited to, sound, light, or temperature emitters. Sound emitters may include, but are not limited to, auditory sound, ultrasound, low frequency sound, and high-frequency sound emitters. Light emitters may include, but not limited to, UV emitters, visible light emitters, and IR emitters. The light emitters may be lasers or LEDs. The emitters may be packaged so that they are modular, so that they can be interchangeably connected to the device 10. These various sensors and emitters and their controllers are commercially available in miniature sizes and may all be readily packaged into a device 10 as large as a cup, for example.

The walls 12 of the coffee mug may be made from a transparent material such as glass or a clear polymer, so that a camera may see through the walls 12 and/or a laser beam may pass through the walls 12. Alternatively, the exterior of the walls 12 may include a reflective film that reflects some exterior light but allows considerable light to pass through the walls 12 so as to reach a camera on the interior of the cup, and/or to permit a laser beam to pass through.

The device 10 may have an optional handle 14. The handle 14 may serve as an antenna, may have USB and/or other ports on it, or may serve other purposes, including simply as a handle 14. The bottom of the device 10 may optionally include a magnetized material, such that the device may be mounted to a surface, such as a metallic table surface 16. The bottom may alternatively include another means for securing the device 10, such as one or more suction cups, adhesive, or other securing approaches known in the art. In some embodiments, the bottom of the device 10 may have USB and/or other ports on it, or may be connected to a power supply.

The device 10 may include a communication nexus that includes one or more communication nodes to operatively connect the emitters and sensors, independently or collectively, to a processing tool that may employ a trainable AI system as described herein. Moreover, the processing tool is presented herein by way of example to a trainable AI system; however, one will appreciate that a more generic processing tool may be substituted for the AI system mentioned anywhere within this description.

The communication nexus may include communication nodes that connect each emitter independently to individual or separate controllers and/or to the processing tool. Similarly, the communication nexus may include nodes that connect each sensor independently to individual or separate controllers and/or to the processing tool. The communication nexus may alternatively or additionally include a communication node that collectively connects multiple emitters and/or sensors to the controllers and/or to the processing tool. Moreover, communication nexus may include one or more communication nodes that connect the emitters to the sensors, emitters to each other, and/or sensors to each other. The connections may convey data or instructions in a single direction or in both directions.

The communication nexus may utilize or connect to a local network via Wi-Fi, Bluetooth, Ethernet cable, or other method for communicating with a network. The device 10 may have one or more ports, such as for USB, flash drive, cables, and/or other accessories. A presently preferred power source is one or more lithium ion batteries, preferably located on the interior of the device 10 in a manner a user may access the batteries for replacement. Lithium ion batteries may be sized to fit into small, irregular spaces. They can also be swapped out and fast charged as needed. The batteries may be rechargeable, such as by a DC power adapter, a USB power source, wirelessly on a recharging pad similar to how many mobile phones are now charged, or other battery charging methods known in the art. It is noted that the wireless recharging pad may have a look and shape of a drink coaster.

In one embodiment, the device 10 includes multiple housings, such as two halves. One half may rest atop the surface, while the other half 20 may rest below the surface. The device 10 is positioned such that a user 22 sitting or standing adjacent to the surface (e.g. a user sitting on a dining room chair) can be scanned by the device 10 with minimal disruption. The lid on one or both halves may include a display screen 24, on which a user 22 or operator may view the body scan.

In another embodiment, the multiple housings may include housings positioned at one more additional locations in a room. These locations may be selected to optimize the possibility that the subject or patient will at some point in time be positioned between the separate housings. In one example, every chair in the room may contain a device 10 or part of a device 10. As noted earlier, these separate housings may have different forms. For example, one housing may look like a cup and another housing may look like a water dispenser. Also, as noted earlier, the devices 10 or parts of devices 10 may have different sets of sensors or stimulus emitters.

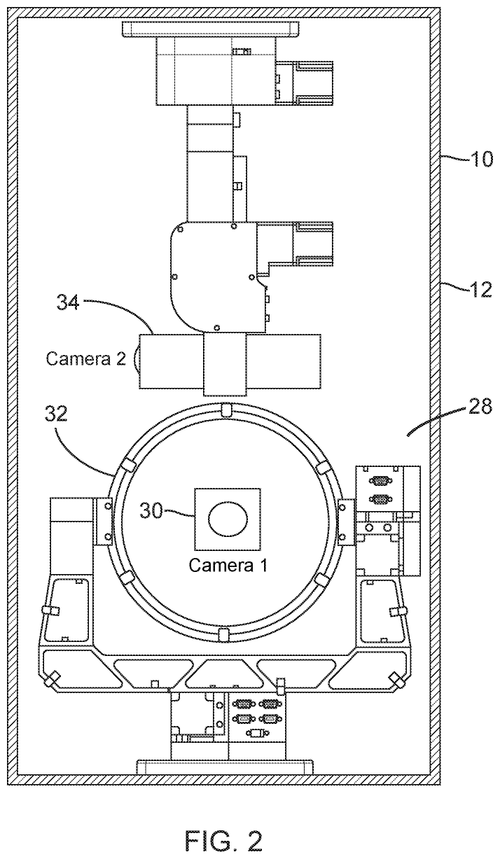

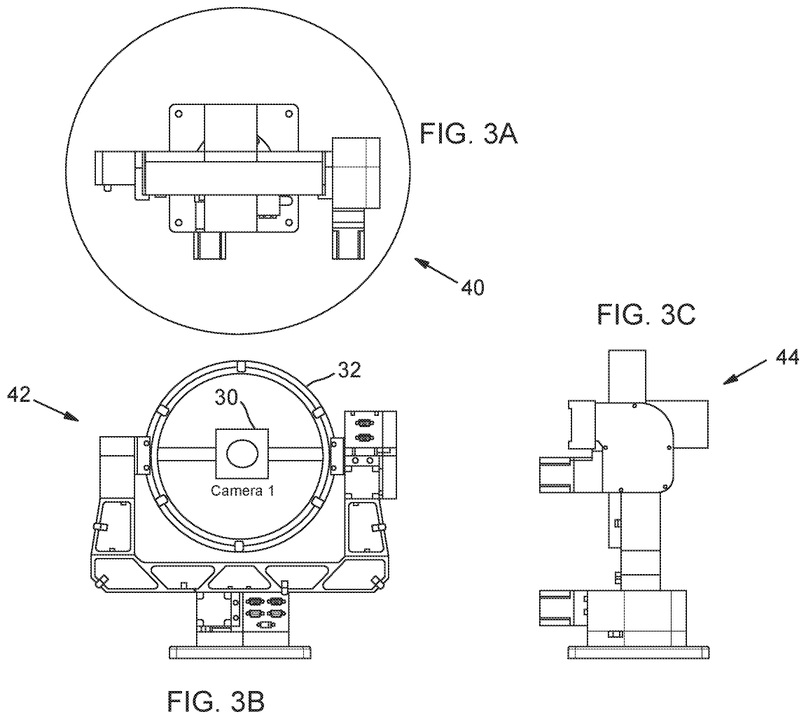

FIGS. 2 and 3 illustrate the interior 28 of the device 10 of FIG. 1. FIG. 2 illustrates an embodiment in which a camera 30 is mounted on at least one gimbal 32 that is rotatable about a single axis. One or more other cameras such as camera 34 may be provided within the device, either on a gimbal or on another type of mount.

The gimbal may be mechanical or motorized, with the embodiments of FIGS. 2 and 3 being motorized. In a gimbal system in which the gimbal is rotatable about a single axis, a single gimbal motor is provided. In an alternative embodiment, a gimbal that is rotatable about two axes is provided.

A three-axis gimbal is a feature of a further alternative embodiment. Powered by three brushless motors, motorized gimbals have the ability to keep the camera level on all axes as the camera operator moves the camera. An inertial measurement unit (IMU) responds to movement and utilizes its three separate motors to stabilize the camera. With the guidance of algorithms, the stabilizer is able to notice the difference between deliberate movement such as pans and tracking shots from unwanted shake. This allows the camera to seem as if it is floating through the air. Optionally, the center ring may be vertically fixed.

FIGS. 3A-3C (collectively FIG. 3) illustrates top 40, front 42, and side 44 views of a gimbal mounting system for a camera.

As previously noted, sensors located inside of the mug will “see” right through the mug material. The portion(s) through which the sensors see is mirrored on the exterior, such that a user 22 will see a reflection from outside the mug. But the film is translucent, and sensors inside the mug may view and/or sense objects that are outside the mug.

At this point, further understanding of aspects of the invention will be facilitated with consideration to concepts of noise, adaptability, error states, adverse user experience, control factors, and algorithms.

Noise

A variety of sources can cause noise. As examples, noise can be created by heat, vibrations, electromagnetic fields, movement, force, vibrations, shock, and other sources. One or more of these may be caused by interaction with other devices according to the present invention located nearby. Noise may be caused through direct contact of the units, connection, and/or proximity. Embodiments of the present invention may include filter circuitry or other means to sense and cancel out such noise.

Adaptability

Various embodiments of the present invention may be durable and adaptable to changing conditions. For example, a unit may be subject to factors such as: different lubricants, high pressure or other forms of cleaning, irregular service intervals, incorrect pressure or flow settings, movement such as being transported by trailer or the like where stability and temperature are not constant, aftermarket parts are used, and different users who use the device in different ways.

Normal degradation of components or materials can lead to decreased performance or even a partial or full loss of system function. As examples, the following may occur in a particular embodiment: corrosion, fatigue, wear, oil degradation, seal ageing, and/or other factors. Alternatively, embodiments of the present invention may be subject to adverse environmental conditions. Such conditions may include water, snow, debris, mud, road salt, dust, stone impact, humidity, ambient temperature (e.g. cold and hot temperatures).

To mitigate effects from the foregoing conditions, it is preferable to employ durable materials, extensive testing, and algorithms that can detect degradation as it occurs so that measures to prevent further degradation and/or system performance may be taken. The measurement may include sensors to detect such factors, and/or algorithms to process information from the sensor and determine corrective measures and/or make design improvements to optimize performance of the system.

Error States

A device according to the present invention may be subject to failure modes that hinder the work and/or function of a device or component. Performance loss may be an effect. Non-limiting examples of error states may be generated by: corrosion, fatigue, difficult service, contamination of fluid, friction, temperature, and/or other conditions adverse to the functioning of the device or component.

Adverse User Experience

A system may work well yet have undesirable effects from an engineering and/or user experience. In the case of a user 22, an undesirable effect may be a result of undesirable feel, audible sense, smell, visual, taste—anything undesirable that may be detected by the physical senses. A user 22 may not like the look of a device 10, for example. Or the device 10 may emit sounds that the user 22 dislikes. It may generate a scent during operation that is undesirable to the user 22. In rare circumstances, the user 22 may experience an undesirable taste in the mouth.

Other adverse user experience factors may include time it takes for a device 10 to complete a procedure, distracting movement of a device 10 and/or its components, shape that does not fit well into the environment in which it operates, and numerous intangible factors that might be best understood by spending time in the shoes of the user 10.

Control Factors

An engineering and/or design team have variables within their control to optimize the function of a system according to the present invention. Non-limiting examples of such variables include: material, dimensions, coating thicknesses, surface finish, sensor type, actuator speed, and many other engineering and/or design variables.

One embodiment of the present invention includes an algorithm that compares such variables to references in a library. The library may be onboard the device 10, partially onboard and partially remote, or fully remote. The algorithm may compare what it finds in the library with variables the engineering and/or design team has selected. The algorithm may make suggestions as to more optimal variable selection, from a system performance standpoint and/or from a user experience standpoint. Reference to the library may help conform a design parameter to best practices, proven design features, user preferences among specific demographics, and other information that may be useful to engineers and designers.

Algorithms According to the Present Invention

Algorithms selected for use in conjunction with the present invention will perform numerous steps. For example, in one embodiment, an algorithm: a) populates a modular unit based on the unit's intended function and/or outputs; b) identifies inputs; c) identifies noise inputs; d) identifies error states; e) identifies unintended inputs; and f) scans and operates a device 10. The foregoing steps may be performed in a different order than the foregoing, in a specific situation.

The algorithm preferably includes countermeasures for encountered noise. This may include filtering, use of active controls, and/or facilitating user intervention. For example, in one embodiment, a user 22 may be provided with the opportunity to select from a variety of modes, such as: a) ignore the noise; b) control or eliminate the noise; c) compensate for effects of the noise; and/or d) minimize the effects. In other embodiments, no input from the user 22 is necessary or desired, and the type and level of control is implemented independent from the user 22.

Returning to the embodiments of FIGS. 1-6, as output the device 10 may transmit data, photos, information about error states and/or undesired side effects, and/or other collected information. An external processing system processes the information, as will be described below. Information concerning the growth of the tumor may be transmitted back to the mug device 10, for display on the lid screen, and/or for reporting in other ways, such as via audio. The device 10 may be equipped with an interactive voice system analogous to Amazon's Alexa, in which the system provides an oral report on command and can answer pre-selected questions.

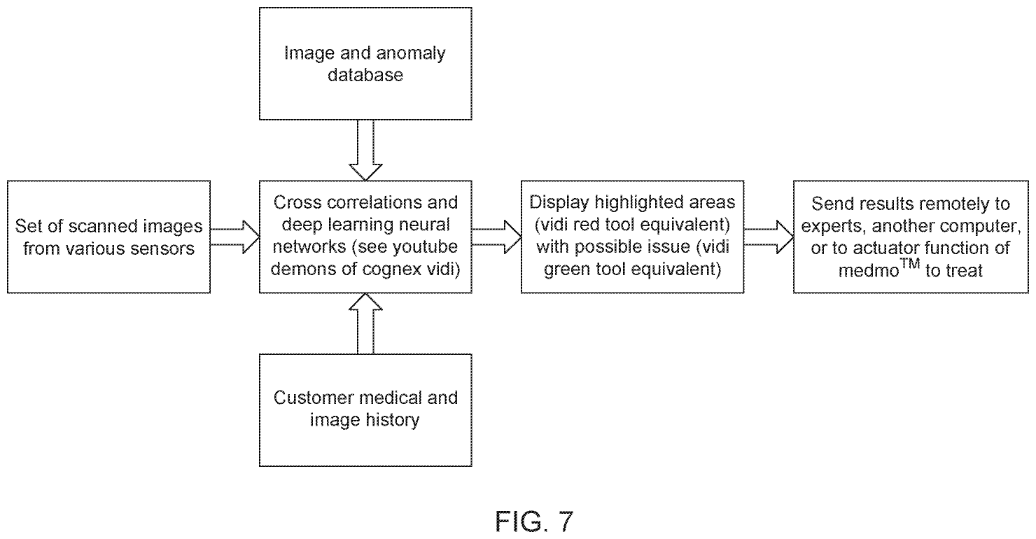

FIG. 7 illustrates a robustness algorithm. The algorithm is to compare data to a library and display final highlighted results on a screen. The display may overlay onto a subject scan.

In a room of multiple people, for example, data about just one person may be desired. Consequently, the unit is adapted to isolate data (e.g. photos) taken of one person the user selects on the touch-screen lid and/or remotely such as from an app on a computer and/or mobile phone.

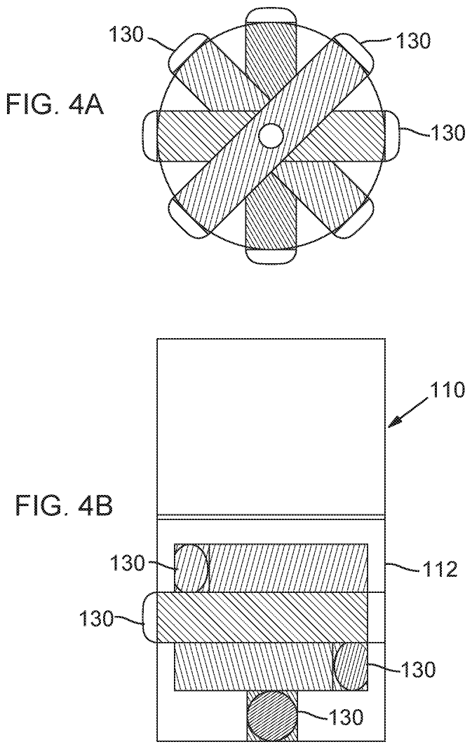

Referring to FIGS. 4A and 4B (collectively FIG. 4), another embodiment incorporates a “spiral” camera concept. Inside the mug, there are 8 miniature cameras and/or sensors, generally represented as cameras or sensors 130. The lenses are snap inserted/press fit after the camera package is dropped into the mug 110. A laser pulse may be made through the same lens. Alternatively, a scanning laser may be located on top of camera array. The cameras and/or sensors are adapted to “see” through the wall 112 of the cup.

In a further embodiment, a fluid seal may be provided on top to simulate having coffee in the mug. The user 22 may have to “drink” it to view the interactive screen, which may be a touch screen or have sealed button controls.

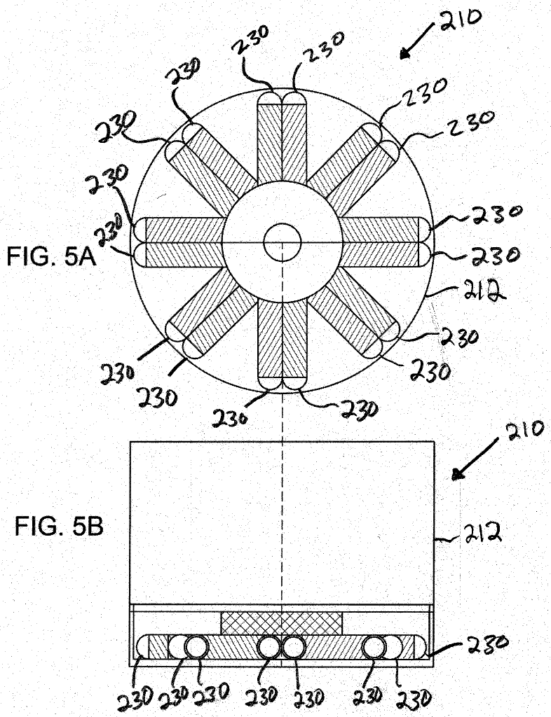

In another embodiment of the portable device 210 illustrated in FIGS. 5A and 5B (collectively FIG. 5), active sensors 230 are employed in sender/receiver pairs. The sensors 230 may be in one plane, and they are rotated and/or they track (such as through a circumferential portion of the wall 212) a moving human, animal, or object's internal organ, structure, or component. The device 10 includes a vertical central “pole” that rotates. The drive motor is at one end, with electronics.

Another variant would be two intertwining spirals (not unlike DNA), where one set is sender-receiver active sensors 230, and the other is passive sensors 230. Rotation can be only as needed, or alternating, to keep track of changes from the last scan—taking turns. The spirals allow for perspective/3D and hologram effect/fill-in for blocked view/mathematical compensation for reflections.

In another embodiment, the device 210 has two sets of active sensors 230, which may be continuously spinning, sometimes spinning, or no movement at all. The sensors 230 may be in a sequential phased array. Each pair would be turned on in sequence, the first turned on right after the last. A three-axis accelerometer and gyro may sense movement of the mug, which is an alternative to the gimbal mechanism previously discussed.

As a further option, the top of the interior mechanisms and electronics may be further down the cup. The mug can then hold a liquid. This does not have to always be surreptitious. It might even hold a liquid medicine or relaxant for the patient being scanned. The device 10 can be scanning the patient while they take the mug, lift to their lips, hold and chat, and even hand it back.

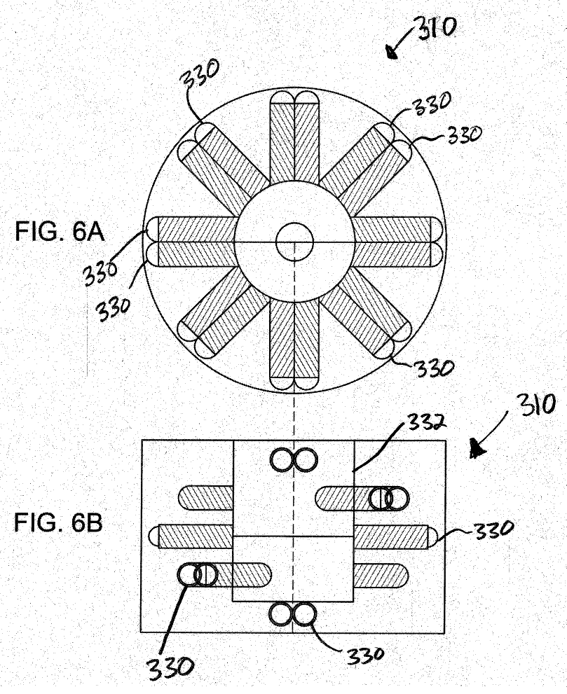

FIGS. 6A and 6B (collectively FIG. 6) illustrate another embodiment of the portable device 310 in which the sensors 330 are provided in a unit 332 that is separate from and attachable to the cup. One will appreciate that these sensors 330 may alternatively or additionally be employed within the device 10. The unit may be snapped or twisted onto the cup, such that the cup may be used to hold liquid. The unit is self-contained with battery and electronics, and the battery may be wirelessly charged with standard cell phone wireless charging, or other charging means.

Considering further aspects of select embodiments of the present invention, the mug will know its location, relative to its initial scan, with the same components inside of a cell phone that convey change in orientation and location. See, e.g., https://www.gsmarena.com/glossary.php3?term=sensors.

Smartphones today come with a wealth of sensors 330 to facilitate a better user experience, provide apps with enhanced information about the world around the phone and provide robust and increased battery life. One is a proximity sensor, which detects when an object is near to the phone. Most commonly used to sense when a phone is held up to the user's ear to turn off the display. This saves both battery life and prevents accidental screen touches.

Other types of sensors 330 are accelerometers and gyroscopes. Accelerometers in mobile phones are used to detect the orientation of the phone. The gyroscope, or gyro for short, adds an additional dimension to the information supplied by the accelerometer by tracking rotation or twist. An accelerometer measures linear acceleration of movement, while a gyro on the other hand measures the angular rotational velocity. Both sensors 330 measure rate of change; they just measure the rate of change for different things.

In practice, an accelerometer will measure the directional movement of a device 10 but will not be able to resolve its lateral orientation or tilt during that movement accurately unless a gyro is there to fill in that info. With an accelerometer you can either get a really “noisy” info output that is responsive, or you can get a “clean” output that's sluggish. When a 3-axis accelerometer is combined with a 3-axis gyro, an output may be both clean and responsive.

Accelerometers are also used to provide ‘steps’ information for a vendor's ‘health’ application.

Another mobile phone sensor 330 is a digital compass. The digital compass that's usually based on a sensor 330 called the magnetometer and provides mobile phones with a simple orientation in relation to the Earth's magnetic field. As a result, your phone always knows which way is north so it can auto rotate your digital maps depending on your physical orientation.

Another common mobile phone sensor 330 is a barometer. The barometer assists the GPS chip inside the device 10 to get a faster lock by instantly delivering altitude data. Additionally, the barometer can be utilized to provide ‘floors climbed’ information to a phone's ‘health’ app. With the advent of more accurate indoor navigation, the barometer can assist in determine what floor a user 22 is on within an airport for example.

Biometric sensors 330 provide levels of enhanced security by capturing and validating human related metrics. Including fingerprint recognition, IRIS (eye) scanning and full facial recognition. Biometric sensors 330 provide a more secure but more convenient way to unlock phones and pay for purchases. Additionally, biometric sensors 330 can be used to collect a user's heart rate and SpO2 (the estimate of arterial oxygen saturation) for use within a vendor's ‘health’ application.

Some sensors 330 may relate to augmented & virtual reality. The highly accurate sensors 330 detailed above, when combined with the powerful CPU & GPU's of modern smart phones, allow very realist and responsive virtual reality applications to be created. When the sensors 330 are combined with a smartphone camera they facilitate augmented reality applications.

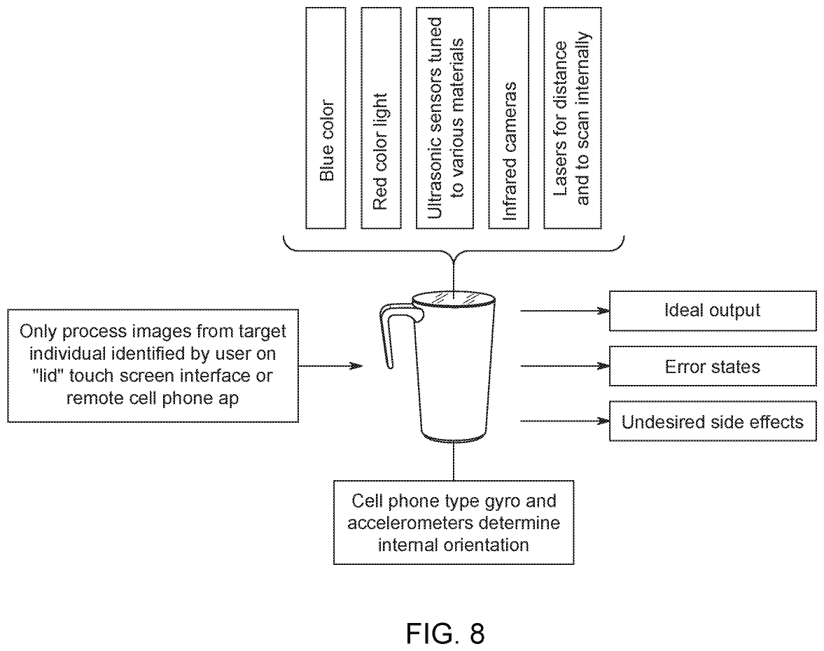

Turning to FIG. 8, one embodiment of a mug device 410 has various characteristics. The device 410 may include a variety of devices, such as various colors of lights, lasers, ultrasonic sensors, infrared and/or other types of cameras, various emitters and the like as desired for particular applications. The device 410 may include gyros, accelerometers, and other sensors 330 as described above. These various sensors and emitters and their controllers are commercially available in miniature sizes and may all be packaged into the device 410. The user 22 may control various aspects of the operation of the device 410 from a touch screen, such as a circular touchscreen at the “mouth” of a cup device 410, or from a cell phone or other remote device that is in communication with the cup device 410. The device 410 may output a variety of outputs, as discussed. The device 410 may also output what is estimated to be ideal output, error states, and undesired side effects from a particular application.

The embodiment of FIG. 8 is configured to gather data about a patient, then transmit the data to an external processing system to predict future growth of a tumor. Graphical and/or other data may then be displayed on a screen, such as on the lid of the mug. The mug may be adapted to provide lighting of any desired type, such as blue light, red light, or other desired illumination, including types of illumination not visible to the human eye, if desired. A variety of sensors are housed within the mug: one or more ultrasonic sensors, an infrared camera, and/or lenses for distancing and to scan internally, devices for determining the internal orientation such as gyro(s) and accelerometers, among other possible sensors.

Systems and Tools for Implementing Algorithms

Considering now an algorithm for scanning and processing, a scanning device generates a set of scanned images from various sensors, such as from the foregoing coffee cup and/or other embodiments. A system then performs cross-correlations and implements deep learning neural networks, drawing from one or more libraries/databases of images and anomalies and/or customer medical and image history. Signals are then sent to the display, to display areas of the body with possible issues. The highlighted areas may be color-coded, such as with red or blue. Results may also be sent to experts located remotely to another computer, or to an actuator function to perform treatment on the user 22.

As background, neural networks are computer programs that are designed to mimic how a human brain operates. They have become the method for how computers learn to perform certain tasks, say recognizing a specific face across different photographs or identifying what a dog is or isn't from a reference set of dog pictures. For further background on neural networks, see “How a Neural Network Helps Manufacturing Inspection,” Cognex Corporation, available at https://www.cognex.com/blogs/deep-learning/what-is-a-neural-network.

Concerning the steps of cross correlation, deep learning neural networks, and displaying highlighted areas of possible interest, a tool may detect defects on complex body parts and surfaces. One step is to locate the object (e.g. a part of the body) of interest. Often the object has complex features. The background may be noisy, poorly lit, low contrast, and may flex or change shape. Consequently, the tool must locate objects despite variations in perspective, orientation, luminance, glare and color by learning from samples provided by the user 22. The system may be trained to find a variety of components that may have a different appearance or vary in size, in order to create an extensive component library. The tool may check multiple feature locations and component types simultaneously, while adjusting to various body layouts.

Alternatively, a sample set of good images and bad images with labeled defects may be used. The system should tolerate normal variations, while detecting true anomalies. For situations where it is difficult to collect images of defects, or if failure modes are unknown, the tool may learn the normal condition by, for example, scanning healthy bodies. After enough samples, it can identify images that stray from this normal appearance.

The tool may also segment areas of an image. The tool can learn to identify areas of abnormality and/or interest. The tool can highlight those areas and, for example, shade them a predetermined color on a display. A commercially available system that performs similar functions is available as the Cognex VisionPro ViDi Red system, available from Cognex Corporation of Natick, Mass. https://www.cognex.com/products/machine-vision/vision-software/visionpro-vidi

Referring to FIG. 7, several steps in one method according to one embodiment of the present invention are illustrated. The sensors gather a set of scanned images. A system such as, for example, as the Vidi system of Cognex Corporation performs cross-correlations, uses deep learning via neural networks or the like, and/or data analysis statistical techniques, drawing on customer data such as medical and image history, a database of images and anomalies, and/or other databases or sources of information. After this step, areas of a sensed image are highlighted, and areas with a possible issue (e.g. identification of potential medical issue) are displayed. In one embodiment, data is transmitted from a processing center back to the device and/or another display such that the user, a medical professional, or others may view the data and make recommendations, prescriptions, treatments, as appropriate.

Systems according to the present invention may also utilize a classifier that can be used to distinguish between different types of objects, identify defect types, and inspect images. Learning from a collection of labeled images, the tool may identify and sort products into classes based on their common characteristics such as color, texture, materials, packaging, and defect type. The tool tolerates natural deviation within the same class and reliably distinguishes acceptable variation from different classes. A commercially available system that performs similar functions is available as the Cognex VisionPro ViDi Green system, also available from Cognex.

As noted, devices according to the present invention often have the capability to multitask. For example, each type of diagnostic is better at identifying/differentiating different kinds of tissue. We also have a pretty good understanding of the degree of confidence we have for each item identified as a candidate for a specific category. How to calculate confidence levels. See https://sciencing.com/calculate-confidence-levels-2844.html. Using GPS as an example, accuracy can be compromised by reflections, partial blockage, lower degrees of correlation with examples in a database, and being too close to the transition between two candidate categories.

Multiple sensors, using compatible software, can get crucial alternate views to estimate 3D dimensions. These can be as simple as one above and below a table, but can also be mounted on a belt, left and right sides of the user's or patient's body (esp. if used for monitoring a condition that could change suddenly). This is more than just a FitBit, for example. But the FitBit has made such monitoring not only accepted, but to be expected.

As a supplement to the foregoing, to understand how every joint changes your orientation, see for example https://www.bhphotovideo.com/c/product/1492980-REG.

It is best that each sensor 130 or 330 have its own ability to collect its own data, then pass that data in an appropriately sampled fashion to the controller board for signal conditioning, drop out interpolations, sensor fusion, false alarm rejection, and cross correlation with library images/features, followed by orientation to a global reference for path planning for any directed action (light/ultrasound/air jet, needle, laser, or blade) in 3D environment (actually 4D, with time variation, as living material moves, so often we need prognostics to predict expansion/contraction, drift/float, and/or dispersion/absorption of tissues).

Regarding the processing units and CPU, relationships can be trained with neural networks, and use correlations to reference cases, some of which might be the actual patient, to track changes and send to doctor. Depending on the features, and complexity of a given set of possible categories, a regular processor may be used for a simple cross correlation, whereas a bank of parallel processors may be needed for deep learning. In the latter case, heavy computer lifting needs to happen on a server. Send the images and//or keep features of those images, wirelessly to that server or to multiple processing facilities around the world hat maintain the best database for the suspected malady. This can readily be scaled up as data accumulates and the device becomes popular, so existing databases adapt their interfaces to share info.

The device and/or external processing system may be programmed with a variety of computer languages, such as R and the higher and lower level languages mentioned in this wiki article. See https://en.wikipedia.org/wiki/R_(programming_language). The “mugs” can act as their own robots, calculating where they are, and where they have been, with respect to a global reference, for example, on the end of a human arm (or static on a table, rotating some sensors with a gimbal/gyro internally).

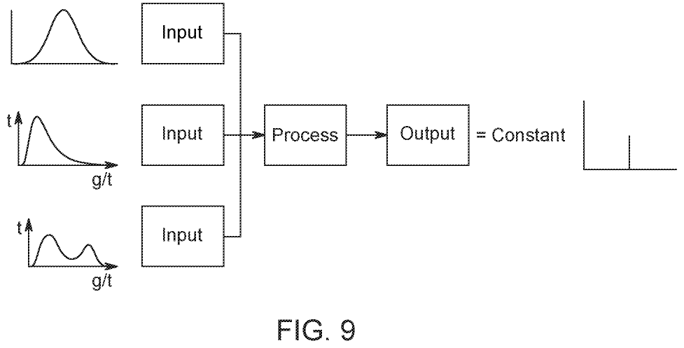

FIG. 9 illustrates conceptually in steps 500 various types of input feeding into a process, which then creates an output.

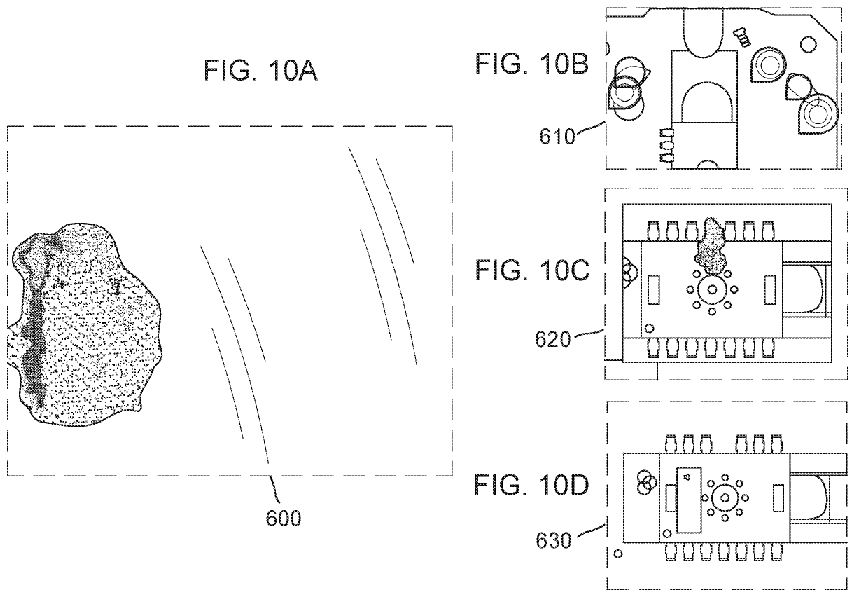

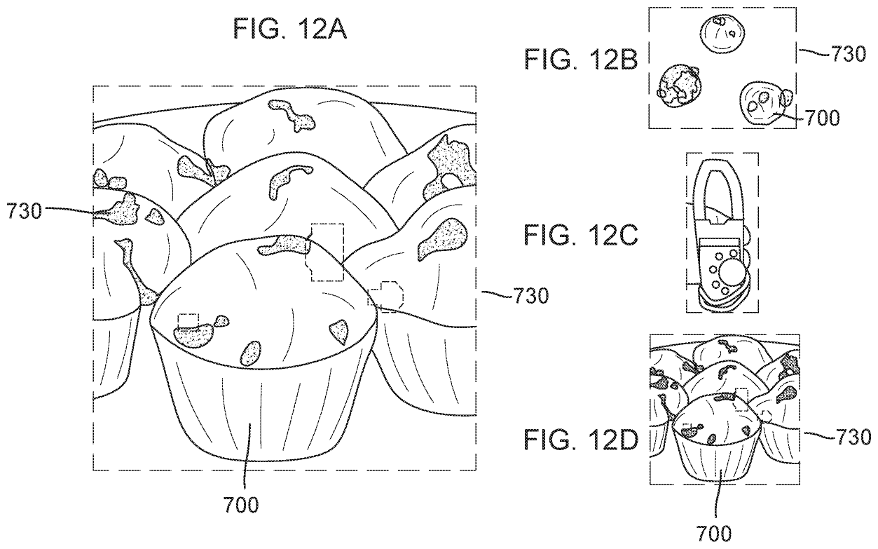

FIGS. 10-14 illustrate an approach for processing images. FIGS. 10A-10D (collectively FIG. 10) illustrate steps in a deep learning system. Steps include classifying an image and/or a portion thereof 600. Individual features of interest are located 610. The located features are analyzed 620. Finally, aspects of the image are read 630. These steps are further explained on the website of Cognex Corporation at www.cognex.com, generally.

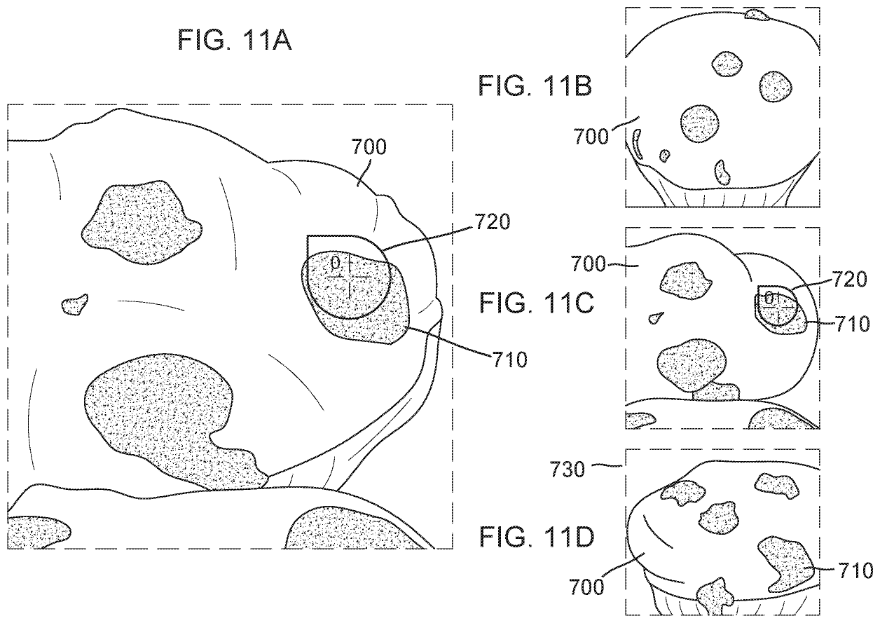

FIGS. 11-14 illustrate aspects of differentiating between similar images, such as distinguishing between blueberry muffins and small dogs. The exterior of the muffins includes portions of spherical blueberries, while the dogs have eyes. The blueberries and eyes are similar in appearance, creating a challenge for a machine vision processing system. Considering FIGS. 11A-11D (collectively FIG. 11), a blueberry muffin has a plurality of blueberries appearing on the surface of the muffin 700. In one approach as described generally in FIG. 10, locations of blueberries 710 are identified as with marker 720. The images are processed with a deep learning algorithm to identify the blueberries as blueberries, as opposed to some other object. This approach can be extended to focus on blueberries on one muffin 700 among a group of muffins 730.

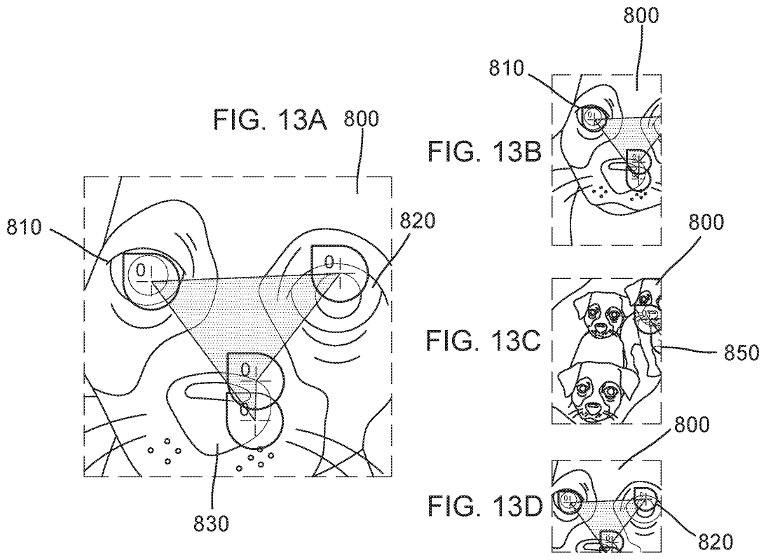

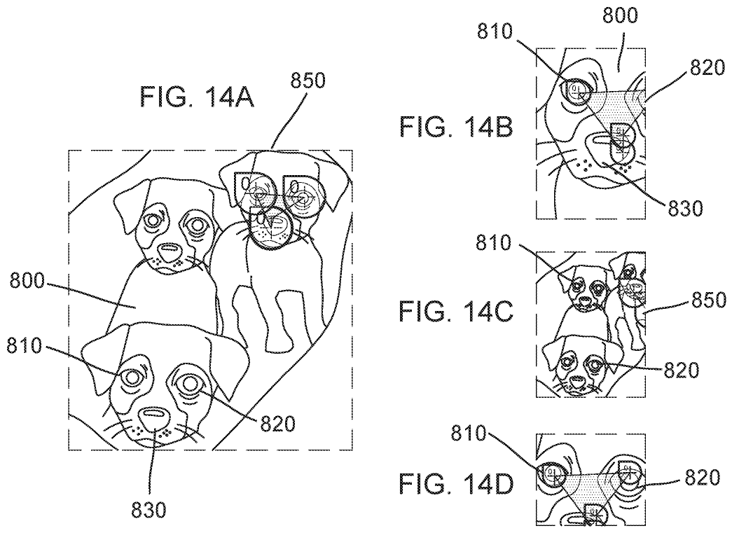

FIGS. 13A-13D (collectively FIG. 13) and FIGS. 14A-14D (collectively FIG. 14) illustrate potential complexity of machine vision, in which eyes 810 and 820 and nose 830 of a dog 800 are to be distinguished from a blueberry muffin. As can be seen, the eyes and nose of the dog appear somewhat similar to blueberries on the surface of a blueberry muffin. And, in a group of dogs 850 (FIG. 14), features of the group may appear similar to a set of blueberry muffins. This problem of distinguishing features from among similarly appearing but different objects has been solved by the inventor using the aforementioned Cognex VisionPro ViDi Red and Green tools, in conjunction with properly populated databases.

Concerning the display screen, in the cup embodiment the read-out screen typically covers many kinds of inputs. The “top” of the mug (where you'd see the “drink”) may have icons that grey out if not being used. For those functions currently in use, there are numbers displaying in real time for critical functions. The menu/settings icon can allow you to scroll through a potentially infinitely long list of items to choose from for display and/or calculations to support the numbers, plots, and images being displayed. You can also project an image on to a wall for showing the patient and/or allowing for more solution. An example may be found at: https://www.amazon.com/Magnasonic-Rechargeable-Hi-Resolution-Presentations-PP60/dp/B016N98GG6

In one embodiment, the screen is round and sits inside of the mug “top.” As examples of screens, see https://www.ebay.com/itm/193153771375 and https://www.ebay.com/i/264191240944.

In one embodiment, the user chooses options around a ring, then scroll choices. The screen can be used not unlike a compass to help orient for better data “fill in” for higher resolution, if desired. The mug may haptically vibrate to help the user tilt with better accuracy. https://www.ebay.com/i/264191240944

Concerning protection from the surrounding environment, one embodiment protects the interior components from heat, desiccation, wear and tear and cleaning. Through the use of seals and/or other means, the unit may be made waterproof. This is optional for some office and home environments in which water is not typically a hazard. But for many uses, waterproofing is desirable. In an emergency, for example, there may be bodily fluids and/or in an unclean environment. The entire unit also needs to be able to absorb/compensate for drops, for instance. The device needs to at least be able to withstand what a human subject can withstand, even if under uncomfortable conditions of extreme temperature, humidity, vibration, acceleration, deceleration, etc.

One approach to cleaning the device is ultrasonic cleaning. All connectors will be encased in the appropriate enclosures to allow this, with just the pins and circumference exposed for interaction with cabling. http://www.budind.com/blog/2014/02/the-mysteries-of-ip-rated-enclosures-explained/

The mug should disassemble in a manner not unlike a Mag flashlight when changing the batteries. Swappable parts should be clearly marked for proper orientation and insertion with a lot of poke yoke (idiot proofing).

The primary intent of this device is to remain portable, but with numerous alterative configurations, able to communicate with and share data with other mugs. Many sensors and actuators can have multiple levels of durability and resolution. Usually, instrument grade versions are not as durable.

Regarding portability, some embodiments of the present invention may be used in various environments, beyond monitoring animals and/or humans. For example, the device may have multiple modes. One mode may be for studying an individual, another mode may be for diagnosing an issue with a machine or vehicle, another mode may be for sensing and processing natural phenomenon such as diagnosing the health of a tree. Many variations are possible. In each case, a portable unit in which are housed one or more sensors, gathers data, has the data processed externally and/or internally as previously described, and results displayed. In some environments, connecting with a network is impractical. Consequently, the device may include onboard memory sufficient to store collected data and/or a removable data card, USB flash drive, or other data storage unit, for later processing.

The device may be adapted to be a medical device for delivering medication. A variety of medications may be delivered to a patient. Jet injection is a preferred mode, via the bottom and/or handle of the “mug,” depending upon where you need to inject. An alternative is to use a needle, although a needle may be more complicated to use than jet injection in this context. As an alternative to injecting medication, the device may be adapted to insert piezo electric meshes. Mesh can be inserted with a catheter needle.

In one medical device embodiment, an anti-blood clotting medicine can be administered from a device that can also defibrillate and produce ultrasound. Defibrillators known in the art are already small enough to be implantable: https://en.wikipedia.org/wiki/Implantable_cardioverter-defibrillator. Known jet injectors are small enough: https://en.wikipedia.org/wiki/Jet_injector. See, also, https://www.healthline.com/health/type-2-diabetes/insulin-jet-injectors#use and, for jet injectors for anti-coagulation: https://www.qegateshead.nhs.uk/sites/default/files/users/user53/gynaeoncology/IL426%20Subcutaneous%20Self%20injection%20for%20anti-coagulation%20treatment.pdf

The device may include accessories, such as for applying energy, ultrasound, injecting or applying medication, and the like. For example, the cup-shaped device may have detachable elements, such as a wand for targeting an area for Transcutaneous Electrical Nerve Stimulation (TENS), defibrillation, or a way to focus a light beam on a certain area of the body or into the eye. Similarly, the device may communicate with and/or control external devices, such medical devices, 3D printers, musical instruments, sound, lighting, temperature-control devices, game gloves, body suits, and other types of external devices appropriate for a particular application. Methods of communication with external devices are known in the art.

Further, in another embodiment, a hand-held device resonates certain substructures of the body. It is often unnecessary to heat the entire body. See, for example, descriptions of magnetic resonance imaging (MM), such as at https://en.wikipedia.org/wiki/Physics_of_magnetic_resonance_imaging and https://en.wikipedia.org/wiki/Functional_magnetic_resonance_imaging

It is noted that in resonating substructures of the body, signals are frequently corrupted by noise from various sources; hence, statistical procedures are used to extract the underlying signal. Sensor fusion, and changes in orientation of the mug(s), can help identify and directly filter out the noise. Noise is that part of the signal which you haven't taken the time to model yet.

That said, microwave ovens can cook at 2.4 GHz at high power. At lower power, we call it Wi-Fi, and Wi-Fi can differentiate objects inside of buildings, with drones outdoors using Wi-Fi. Bluetooth is usually 2.4 GHz right against our heads, but it really doesn't have enough power to penetrate the skin. In between, we could heat up certain target organs. See, e.g., https://wade4wireless.com/2014/02/01/rf-exposure-to-humans-and-much-more/.

In one approach, a particular organ that resonates at a particular frequency may be stimulated by resonating a seat haptically, via stimulation from the mug or in conjunction with a simple unbalanced motor. For more on whole body vibration, please see https://en.wikipedia.org/wiki/Whole_body_vibration

A device according to the present invention may resonate across several frequencies to see how various structures respond, in a similar way.

The present invention may include using sound to move objects. The device may include a piezoelectric crystal speaker. For background on moving things with sound, see https://www.youtube.com/watch?v=L5fVFA2sWt4. For background on making a piezoelectric crystal speaker, see https://www.youtubecom/watch?v=R7zjfaPKMSE.

Detecting Physiological, Emotional, and/or Other Responses to Stimuli

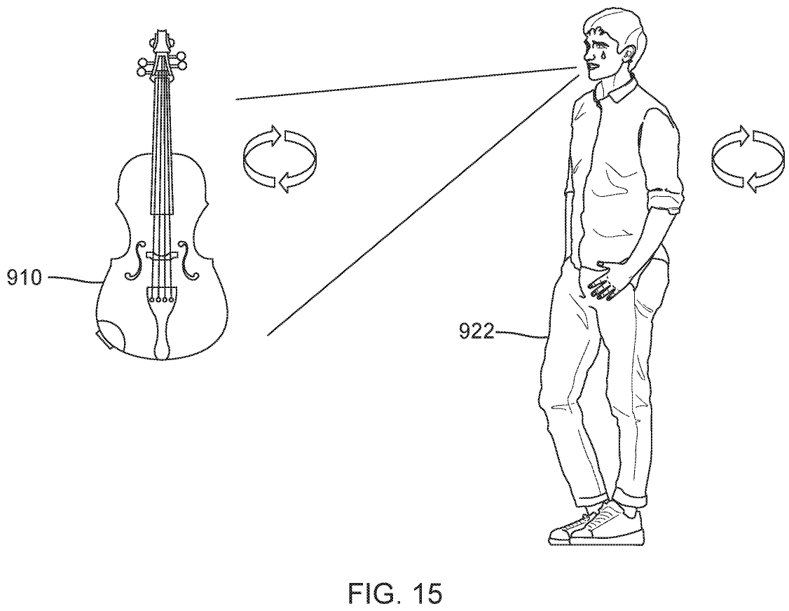

Turning now to FIG. 15, a sensing device 910 is configured as a violin or other musical instrument. The device 910 is an assessment device that identifies and modifies physiological responses of a subject 922 in response to stimuli. For example, an assessment device, such as a musical modulation optimizer (herein referred to as “Musemo™”) can identify and modify physiological responses in response to musical stimuli. The physiological response may be one or more of heart rate, blood pressure, pupil diameter, facial expression, tearing up, swaying to music, change in seating position, etc. A processing tool can determine whether physiological responses indicate psychological or emotional states in response to musical stimuli. The processing tool may employ an AI system and the processing tool may be presented herein by way of example to a trainable AI system; however, one will appreciate that a more generic processing tool may be substituted for the AI system mentioned anywhere within this description.

The device might also, or alternatively, identify and modify psychological or emotional states include sorrow, joy, arousal, confusion, etc. An AI system employs a neural network to determine whether physiological responses indicate psychological or emotional states in response to musical stimuli.

By way of overview, Musemo™ can take feedback from:

1. Devices as generally described in previous sections: Human/animal/plant/machine measured/changes/locations/resonances between various independent sources of: overall body and/or internal organ/structure size, temperature, moisture, heart rate, respiration, chemistry, emissions, color

2. Entire organism's movements, nervous jitter/tapping, swaying, dancing frequency, sounds they make, independently—and in interaction with others (other living beings or machines, and of course its interactions with the Musemo™ itself)

3. Other Musemo™-type devices

4. Other Musemo™-type devices

5. Other devices in communication with the Musemo™ device

Musemo can actively:

1. Play music, both melody by hand from the human, and melody and/or harmony generated like a player-piano-violin and/or completely synthesized notes and/or prerecorded music/singing—continuous/sampled . . . .

2. Speed up and/or slow down the sounds to encourage a stronger resonant response from the Human/animal/plant/machine (entity) of interest. Methods of modulating frequency are well known. https://www.youtube.com/watch?v=V-Cj07Afzrw

and https://www.youtube.com/watch?v=ZgMaBBwcI_4.

Considering the Musemo™ concept broadly, music can have a profound effect on a human, even triggering intense emotional responses. There are many documented instances of music—sometimes in conjunction with video or other media—inducing crying in viewers. For example, a scene in a television show might have music composed to trigger an emotional response, in conjunction with a visual theme that also tends to elicit feelings in the viewer. An example is a scene in which a baby lies near a dying mother as music plays. See, e.g., https://youtu.be/6M9SaBbut8A Even a video of a horse dancing to music in a freestyle competition can elicit an emotional response in a viewer. See, e.g., https://youtu.be/zKQgTiqhPbw

Turning again to FIG. 15, a violin 910 is provided having a variety of sensors, microphones, actuators, and/or other devices including lighting devices and/or lasers. Optionally, an initial prototype may be created using 3D printing and tested. If the device fails, the design may be altered, and a new prototype printed. Once tests on a prototype are successful, a wooden version may be created.

In one embodiment, sensors in the violin have a push button that adds a concert accompaniment from an onboard music synthesizer-type device, which triggers various natural frequencies. The system continues this until images show the customer starting to respond favorably. Examples of favorable response can be facial expression, tearing up, swaying to music, crying, changing seating position, or other physiological responses. Recurrent and radial basis function neural networks interpolate the ideal natural frequency to trigger desired reactions in the customer.

Conversely, if desired, the system can cycle through a pattern of frequencies until it detects a negative reaction in the user, such as frowning, pursing the face in anger, nervousness, shifting about in a seat, pacing, or the like.

The device can be operable to manipulate, such as enhance or diminish, a natural response to the musical stimuli, as music can be readily manipulated to resonate with a person's natural frequencies, such as for crying, joy, or pleasure. As an alternative to music, the device may play binaural beat patterns and observe user response to a particular binaural beat. This is consistent with the intent of binaural beat compositions, intending to elicit sleepiness, relaxation, concentration, energy, or other states, as desired. However, not everyone responds to music, sounds, binaural beats, and the like in the same way. Consequently, the present system may monitor the listener for face reactions, body language, and other factors to indicate if the music and/or other stimulation is triggering the desired effect.

William Pielemeier used seat vibration to determine human resonant modes, so he could design a seat that didn't excite those modes. See “A high-resolution time-frequency representation for musical instrument signals,” The Journal of the Acoustical Society of America 99, 2382 (1996). This article is herein incorporated by reference. The article may be found at https://asa.scitation.org/doi/10.1121/1.415426. See also U.S. Pat. No. 5,618,995, which is herein incorporated by reference.

One embodiment of the present invention is a system and method for exciting those modes in the human eardrum and other organs. In one embodiment, a device such as a violin or mandolin can be adapted to be self-tuning. The device can serve as a demonstration tool to show that animals, such as people, can be precisely manipulated as electrochemical saltwater radios.

In one embodiment, the device may be shaped as a string instrument such as a violin and is made with 3D printing. One example of 3D printing a violin is disclosed at https://contest.techbriefs.com/2016/entries/consumer-products/6678 and https://www.3d-varius.com. Sensors and electronics, such as those previously described, may be located within the violin.

In another embodiment, the scanning technology can be used to analyze the sounds and structure of a Stradivarius violin. The feedback can be used to make iterative 3D printed structures that sequentially are better at reproducing the sound. Additionally, an intelligent bow can be designed to achieve different sounds at different angles.

In another embodiment, a game glove or body suit can be provided to provide stimulus and feedback.

The invention encompasses not only a multi-sensor, but also multi-actuator: light, ultrasound, heat, and sound/music can all be manipulated to excite a resonance in the patient that can be used for diagnostics. The patient can also tell the examiner if the resonance results in emotional changes, pain, or pleasure.

Numerous variations on the foregoing concepts fall within the scope of the invention. In one embodiment, the device may direct various colors of light, ultrasound, and other active sensing devices toward the targeted patient, then measure the reflected signals.

The device may also passively sense parameters, such as temperature, and smells, and movement, both before the active sensors are engaged, and compares to how the patient's body reacts to the active sensors themselves.

In some embodiments, the device can communicate with other “modular” (swappable sensors) devices to broaden the data that can be compared to the database for one or more matches to known conditions. These other devices can have different sensors, or be duplicates, and can compare data while scanning the same patient, or another, who might be acting as a control, or a potential fellow victim of a malady.

The device may adaptively vary its active sensors to achieve a resonant mode in a given organ. This can aid in diagnosis and can also be used to mitigate pain by relieving tension and/or triggering the body's release of endorphins or other chemicals/hormones. The resonant mode of an organ is observed by the sensors (active or passive) tracking a change in the organ. This resonance can be motion based, chemistry based, odor based, or sound based, just to name a few possibilities.

In one embodiment of the present approach, multiple organs can be observed and modulated at the same time. As one example, oximetry measurement of oxygen in the retina of the patient's eye may be measured and compared to behavior of an active sensor stimulated potential bleeding fibroid elsewhere in the body. The Medmo™ device may also be employed to detect oxygen saturation in the retinal blood vessels to assess diabetic retinopathy, glaucoma, or a retinal vascular occlusion.

One embodiment of the present invention may be wired to, or wirelessly interact with, prosthetic devices to stimulate the patient externally, such as with a Fitbit wristband, an instrumented glove, or instrumented body suit. These prosthetic devices can also directly measure temperature, blood pressure, pulse, sweat chemistry, and odors directly.

Further, an ultrasonic sensor can display the interfaces between objects of differing density when touching the exterior of that object.

Considering other aspects of specific embodiments of the invention, one aspect of this disclosure relates to a portable scanning device useful for detecting medical conditions and/or personal threats such as surveillance devices or injury causing devices such as bombs. Moreover, the Medmo™ device may be employed to detect physical objects on a person or animal and/or may be employed to diagnose mechanical systems using the stimulus emitters and the sensors. The trainable AI system may employ object recognition technology such as available in some checkout scanning systems; however, the Medmo™ device may utilize more than optical data.

An aspect of this disclosure relates to a means to detect potentially life-threatening conditions that are normally detected via x-ray, but are often found in the abdomen, which tends to excessively absorb x-ray radiation. One specific application, that could fit into one of these modular units, is to detect and locate bleeding fibroids. They can be pulmonary (say, left chest), uterine (lower abdomen), or other places in the body of a human or animal (or possibly even a plant). There have been many cases of women in their 50's and 60's suddenly bleeding heavily internally. There are several ways to detect them without having to overdo x-rays as discussed herein and shown in FIG. 10. The Medmo™ device may also locate a mass and differentiate it from normal tissue. For example, Medmo™ device utilizing the trainable AI system may differentiate an ovarian cyst from a normal follicle that is about to release an egg.

Another aspect of this disclosure relates to its modularity for handheld use, critical to remote geographical locations, scanning humans trapped in tight spaces, or for scanning unknown to the subject being scanned. One type of handheld packaging for Medmo™ could employ a handheld tricorder medical device such as disclosed at http://www.tricorderproject.org/about.html (the text, the design and/or capabilities of which are herein incorporated by reference). There are seven patent applications that list one or more of Basil Harris, George Harris, Edward Helper, and Constantine Harris as an inventor in connection with a device referred to as DXTER™. These patent applications are incorporated herein by reference. One or more of the functionalities of the DXTER™ device as disclosed in these incorporated patents can be included in the portable assessment device.

However, this disclosure also proposes a mug (or mug-shaped) type design because it is non-threatening to the subject, easy to hold by the user, and functionally suited to internal sensor and mirror rotation.

In some embodiments, one can vary the scanning via https://velodynelidar.com/newsroom/how-to-change-laser-angle-and-fov-v1p-16/ (the text, the design and/or capabilities of which are herein incorporated by reference). Moreover, U.S. Pat. Nos. 8,767,190, 9,983,297, 10,018,762, 10,048,374, and 10,197,669 are herein incorporated by reference. One or more of the functionalities of the scanning systems disclosed in these incorporated patents can be included in the portable assessment device.

The devices 10, 110, 410 herein (regardless of whether they are mug shaped, tricorder shaped, instrument shaped, or other shaped) may generically referred to as a “Medmo™” device. In general, a Medmo™ device may passively collect data that it observes, may actively scan a patient without substantially changing the patient, directly stimulate with a laser or ultrasound to try to get a response, and gain enough information to potentially treat the patient right then and there, and potentially treat the patient. Moreover, a “Musemo™” device, which is a variation of may “entertain” the patient with music/other sound effects (and possibly fragrance) in order to calm the patient down for better data collection, change the states of a patient (emotional and/or organ vibration/resonance) to generate more data, gain enough information to potentially treat the patient right then and there, and potentially treat the patient. As later described in greater detail, a Musemo™ device may have the ability to play music (or other sounds, signals or any type), then observe a change in the subject (human, animal, plant, machine), then modify its output in response. This process may be provided as a single application or as or a feedback loop until a certain response is achieved. The Musemo™ device may for example initially observe restlessness and pain in an individual, but may end up observing calm and tranquility in the individual. The Musemo™ device may resonate a target organ to make it easier to diagnose an issue, may observe a change of state (more stressed, lees stressed) in and of itself, and may provide the data points for additional analysis.

In some additional, alternative, or selectively cumulative embodiments, this Australian researcher's work can be incorporated into this device. If the magnetic resonance can be focused, there can be less of an impact on the subject. The Medmo™ device may be operable to be hooked up temporarily to a larger device that would provide the means to accumulate enough energy to be effective. “We have a unique opportunity to utilize a new minimally invasive therapy for symptomatic uterine fibroids called Magnetic Resonance guided Focused Ultrasound (MRgFUS) for fibroid-related research.” https://www.thewomens.org.au/research/research-centres/womens-gynaecology-research-centre/research-themes/wgrc-abnormal-bleeding-uterine-fibroids (the text, the design and/or capabilities of which are herein incorporated by reference). For example, the technology employed in the ExAblate 2000 (InSightec Ltd., Haifa, Israel) combines magnetic resonance imaging (MRI) with high-intensity focused ultrasound to destroy tumors non-invasively. U.S. Pat. Nos. 9,623,266, 9,814,909, 9,852,727, 9,934,570, and 9,981,148 are herein incorporated by reference. This Magnetic Resonance guided Focused Ultrasound (MRgFUS) technology can be adapted to be employed in and/or with the portable assessment device.

This Ted talk describes the colored regular light that can be used by the Medmo™ to locate tumors. https://www.ted.com/talks/mary_lou_jepsen_how_we_can_use_light_to_see_deep_inside_our_bodies_and_brains/transcript?language=en (the presentation, the design and/or capabilities presented therein are herein incorporated by reference).

Another application involves a blue laser aimed into an eyeball to detect heavy internal bleeding. The machine Mark J Rosen (Pulmonary Medicine, Mount Sinai Doctors Faculty Practice, 36 West 60th Street, New York, N.Y. 10023) is evaluating currently costs $500K, but if you turn the power down on this Keyence parts inspection laser, with its own control board, so you don't hurt the eye, and you have the same functionality in a small package, and quite affordably.

https://www.keyence.com/products/measure/index.jsp (the text, the design and/or capabilities of which are herein incorporated by reference).

The “Retinal oximeter” was first developed in 2002 by Chris Gregory. https://www.newscientist.com/article/dn2363-look-in-the-eye-reveals-internal-bleeding (the text, the design and/or capabilities of which are herein incorporated by reference). http://eyewiki.aao.org/Retinal_Oximetry (the text, the design and/or capabilities of which are herein incorporated by reference). In conjunction with this disclosure, the hardware of this technology can be used to scan older women at high risk for bleeding fibroids.

Older women are not the only patients that will benefit. Coal miners are dying from black lung, from the quartz dust in the coal mines. They often suffer from https://pulmonaryfibrosisnews.com/2017/05/25/twelve-facts-about-pulmonary-fibrosis-prognosis-and-life-expectancy/2/ (the text of which is herein incorporated by reference).

Brain and other tumors are excellent to scan frequently for changes after a surgery might not have eliminated all cells. These tumors can regrow and cause issues as early as 6 months to as late as a decade later. https://www.nbc4i.com/news/u-s-world/an-8-year-old-boy-celebrates-after-beating-stage-4-brain-cancer/1632910704 (the text of which is herein incorporated by reference).

These scanning devices can also be used as tools to ameliorate disease. For example, a Medmo™ ultrasound scanner can be used to treat Alzheimer's disease. See https://www.wvnews.com/statejournal/news/historic-breakthrough-wvu-rockefeller-neuroscience-team-first-to-use-ultrasound/article_b9951ba2-19ba-54ba-8e1c-0096fb4824bc.html

Ultrasound technology can be packaged into handheld devices. For commercially available handheld ultrasound devices, see https://www.bing.com/shop?q=handheld+ultrasound+devices&FORM=SHOPPA&originIGUID=E018626F2D6B4C4B98E5335F6F8F51BA

The Medmo™ device can be also packaged to include defibrillator technology. For commercially available handheld defibrillator technology, see https://www.amazon.com/HeartStart-861284-Philips-Home-Defibrillator/dp/B00064CED6

The Medmo™ device can also be packaged to include “TENS” technology. For commercially available handheld “tens” technology see https://www.bing.com/shop?q=handheld+tens+unit&qs=n&form=SHOPSB&sp=−1&pq=handheld+tens+unit&sc=0-18&sk=&cvid=5E57EA01AEB34F4EAF0E946E67363840

If someone is getting cold from poor circulation, you could actually warm them up via localized resonance activity. If you cause someone to resonate, their body temperature will almost always go up. Infrared sensors can actually warm you up if you continue to use them a long time. Of course, you want to monitor so they don't get too hot. Also, human body movement can also recharge a Medmo™ worn on the person.

These scanning devices can also be used to scan and resonate inanimate objects and mechanisms, similarly to the way they can scan living things. For example, the scanning devices can utilize Flir™-like technology to identify hazards, such as gas leaks, or even occluded objects, such as pipes or wiring behind walls in the context of remodeling projects. See for example, “No, really. You can see through walls using drones and Wi-Fi” https://www.theregister.co.uk/2017/06/20/drones_and_Wi-Fi_see_thru_walls/and U.S. Pat. Nos. 5,345,304 and 8,659,664, which are incorporated herein by reference.

These different scanning technologies (emitters and sensors) can be packaged together in arrangements that optimize their performance. They can share power supply, master controller, communications nexus, and external connections to external devices, power cords, Ethernet, etc. They can also be constructed as modular add-ons that are adapted to connect in a specific manner so as to functionally integrate with any needed internal systems.

The Medmo™ scanners should be carefully calibrated to avoid wave interference that could cause extreme amplitude issues in eardrums, organs, music, heat, hormones, etc. See https://en.wikipedia.org/wiki/Wave_interference. The AI system can be utilized to assist with identification of off-calibration and correction thereof.

Considering other potential aspects of a Medmo™ device, the be paired off with an ultrasound probe like this: https://www.fastcompany.com/1725155/ultrasound-scans-your-baby-now-available-smartphone. Medmo™ can combine this with Wi-Fi to scan for objects inside of the human body that vary in density, from foreign objects (swallowed object/bullets) to possibly cancerous/fibroid growths of different density if the Wi-Fi is tuned properly and in conjunction with/trained by ultrasound. https://www.youtube.com/watch?v=fGZzNZnYIHo. In some instances, Wi-Fi can be used to see through walls. Medmo™ can use a 40 Hz oscillator to stimulate peoples' brains to stop/reverse Alzheimer's. https://www.mayoclinic.org/diseases-conditions/alzheimers-disease/expert-answers/music-and-alzheimers/faq-20058173 and https://gammalighttherapy.com/collections/40hz-light-devices/products/gamma-40-hz-light-therapy-kit

One goal of select embodiments of the Medmo™ devices is to use cheaper, more robust, readily available, easy to replace, simple variations of current medical technology to achieve the same results. https://store.synthrotek.com/555_Timer_Oscillator_Kit

In some embodiments, Medmo™ can vary the colors used to diagnose, based on brain resonance, what parts of the brain are responding to varying degrees to the 40 Hz. Medmo™ can also oscillate other actuators (not just light) to vibrate at 40 Hz (or other frequencies, as harmonics) to resonate other organs/muscles/nerves/skin/bones to stimulate healing (we do this with ultrasound and STEMS now). Medmo™ can vary at other frequencies.

Medmo™ may have modular plug and play attachments that will automatically recognize each other and activate sensor fusion (using neural networks) software to co-locate and co-target the same tissues/tumor/cut/tendon pull and work in synchrony (same time as resonance, or alternating their effects—one to aid secretion, the other to resonate to better absorb the hormone just secreted).

These devices (paired up in one handheld, or two working in synchrony) can not only play music to help Alzheimer's patients (or any other mental condition with brain function issues/damage) not only find an alternate path to information (music is stored in multiple parts of yours brain, and accessing one memory can stimulate other memories near it)—but Medmo™ can pinpoint where the activity is, and resonate it to trigger even more response. It can also direct hormones/drugs to act in that location by vibrating at a frequency that triggers the hormone/medication to activate/combine at that spot.

Some embodiments of a Medmo™ device can help Down's Syndrome patients better transfer short term to log term memory (their major issue—and why we try to push as much info into them when they are very young near 3 years old, when it's still easiest to help them retain information). Medmo™ can help locate where to inject stem cells, and then stimulate stem cells to stay where they are and start dividing in a specific spot (brain, damaged parts of the body, including thinning walls of an artery).

Some embodiments of a Medmo™ device can be used to slow internal bleeding at the scene of an accident—inject the clotting drug directly into the area (chest) and then stimulate it to act. This can be a blood clot and it can also be the unfolding and wrapping of a piezo electric mesh that was just injected into the body. This mesh can not only be metal to bend with electrical zapping optionally from the large power source/battery that comes with the defibrillator, it can also be applied around a tiny clot cloth that was injected via catheter needle that swells up once inside the body (blood itself!) https://www.ebay.com/i/163764749516.

Some embodiments of Medmo™ devices can be used to look at arterial damage progression just before and just after CPR/defibrillator application, and then move to mitigate holes that broke open from shocking near the clot that caused the heart attack stroke to begin with. https://www.osha.gov/Publications/3185.html

Considering further background, concepts utilized in driving neural network patents and later lane departure warning patents by Dean Pomerleau can be adapted for use with the portable assessment device: images fed into a neural network; hidden units are mini images that can be reused elsewhere to cross correlate mini features to determine if feature is a lane, another vehicle, etc.; original neural network's output was a steering vector; mixture of experts chose what actual steering command to actually deploy; and from the outputs of competing neural networks tuned to various driving surfaces. U.S. Pat. Nos. 5,448,484 and 5,091,780 are herein incorporated by reference.

Analyzing images for details is also disclosed in http://www.tricorderproject.org/papers/jansen_fiacconi_gibson_2010_neonate_saccades.pdf, which is herein incorporated by reference.

One will appreciate that many forms of artificial intelligence (AI) can be used instead of, or in addition to a neural network (which is for convenience in this disclosure is considered to be a specific form of AI). These might include probabilistic techniques such as Bayes or Markov algorithms, kernel methods (like SVM, decision trees/random forest, Gaussians, PCA . . . ), reinforcement learning that can have nothing to do with artificial neural networks, artificial reasoning a.k.a. “good old fashioned AI,” many path-planning and intelligent control-systems methods that correspond to “classical AI” (not the same as GOFAI), Alife (swarms, cellular automata . . . ), agents and chaos systems, and/or any algorithm or group of algorithms that optimize a value function (reinforcement learning and linear dynamic programming).

The Medmo™ device may include a communication nexus between the sensors and the AI processing tools. Some or all of these AI processing tools may be positioned within the Medmo™ device itself, such as in an “onboard” computer, and/or in communication with a master controller. One will appreciate that the communication nexus may also use or rely on a wired or wireless connection to nearby or offsite AI processing tools.

Selectively cumulative embodiments are embodiments that include any combination of multiple embodiments that are not mutually exclusive.

Additional aspects and advantages will be apparent from the following detailed description of example embodiments, which proceeds with reference to the accompanying drawings.

Some embodiments do not use CAT SCAN x-rays or MM MAGNETIC imaging, as these are both risky for some individuals even once, and for all individuals, multiple times. Medical personnel can then determine if these machines are necessary after examining diagnostic results from the device and system.

### CONCLUSION

Unless otherwise expressly stated in the drawings, the sizes, positions, etc., of components, features, elements, etc., as well as any distances therebetween, are not necessarily to scale, and may be disproportionate and/or exaggerated for clarity.

The terminology used herein is for the purpose of describing particular example embodiments only and is not intended to be limiting. As used herein, the singular forms “a,” “an” and “the” are intended to include the plural forms as well, unless the context clearly indicates otherwise. It should be recognized that the terms “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise specified, a range of values, when recited, includes both the upper and lower limits of the range, as well as any sub-ranges therebetween. Unless indicated otherwise, terms such as “first,” “second,” etc., are only used to distinguish one element from another. For example, one element could be termed a “first element” and similarly, another element could be termed a “second element,” or vice versa. The section headings used herein are for organizational purposes only and are not to be construed as limiting the subject matter described.

Unless indicated otherwise, the terms “about,” “thereabout,” “substantially,” etc. mean that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art.

Spatially relative terms, such as “right,” left,” “below,” “beneath,” “lower,” “above,” and “upper,” and the like, may be used herein for ease of description to describe one element's or feature's relationship to another element or feature, as illustrated in the drawings. It should be recognized that the spatially relative terms are intended to encompass different orientations in addition to the orientation depicted in the figures. For example, if an object in the figures is turned over, elements described as “below” or “beneath” other elements or features would then be oriented “above” the other elements or features. Thus, the exemplary term “below” can, for example, encompass both an orientation of above and below. An object may be otherwise oriented (e.g., rotated 90 degrees or at other orientations) and the spatially relative descriptors used herein may be interpreted accordingly.

Unless clearly indicated otherwise, all connections and all operative connections may be direct or indirect. Similarly, unless clearly indicated otherwise, all connections and all operative connections may be rigid or non-rigid.

Like numbers refer to like elements throughout. Thus, the same or similar numbers may be described with reference to other drawings even if they are neither mentioned nor described in the corresponding drawing. Also, even elements that are not denoted by reference numbers may be described with reference to other drawings.

Many different forms and embodiments are possible without deviating from the spirit and teachings of this disclosure and so this disclosure should not be construed as limited to the example embodiments set forth herein. Rather, these example embodiments are provided so that this disclosure will be thorough and complete, and will convey the scope of the disclosure to those skilled in the art.

The terms and descriptions used above are set forth by way of illustration and example only and are not meant as limitations. Those skilled in the art will recognize that many variations, enhancements and modifications of the concepts described herein are possible without departing from the underlying principles of the invention. For example, skilled persons will appreciate that the subject matter of any sentence or paragraph can be combined with subject matter of some or all of the other sentences or paragraphs, except where such combinations are mutually exclusive. The scope of the invention should therefore be determined only by the following claims.

1. A detection system including a portable device configured for detecting medical conditions or physiological reactions in an animal body, comprising:
a housing shaped to disguise the detection system, wherein the housing has an interior that is at least partly bounded by an exterior wall portion, and wherein the exterior wall portion comprises a material that is opaque or reflective when viewed from the exterior but is translucent or transparent when viewed from the interior of the device toward the exterior wall portion;
multiple stimulus emitters within the portable device, wherein the multiple stimulus emitters include first and second stimulus emitters, wherein the first stimulus emitter is operable to emit a first type of stimulus toward the animal body, wherein the second stimulus emitter is operable to emit a second type of stimulus toward the animal body, and wherein the first and second stimulus are of different types;
multiple sensors within the portable device for obtaining body data associated with a medical condition or physiological reaction in the animal body, wherein the multiple sensors include first and second sensors, wherein the first sensor is operable to obtain a first type of body data, wherein the second sensor is operable to obtain a second type of body data, and wherein the first and second types of body data are different, and wherein the first sensor or the second sensor comprises a camera; and
a communication nexus that includes one or more communication nodes within the portable device to operatively connect the sensors, independently or collectively, to a processing tool for processing the body data to identify the presence of one or more medical conditions or one or more physiological reactions in the animal body, wherein the processing tool comprises a trainable artificial intelligence system (AI system).

2. The detection system of claim 1, wherein the portable device is configured to be held by a hand.

3. The detection system of claim 1, wherein the housing is shaped to resemble a piece of drinkware or a musical instrument.

4. The detection system of claim 1, wherein the portable device includes multiple stimulus emitters within the portable device, wherein the multiple stimulus emitters include first and second stimulus emitters, wherein the first stimulus emitter is operable to emit a first type of stimulus toward the animal body, wherein the second stimulus emitter is operable to emit a second type of stimulus toward the animal body, and wherein the first and second stimulus are of different types.

5. The detection system of claim 4, wherein one of the stimulus emitters emits one or more of UV light radiation, visible light radiation, infrared light radiation, microwave radiation, radio sound radiation, and ultrasonic radiation, or wherein one of the stimulus emitters is a laser.

6. The detection system of claim 1, wherein one of the sensors comprises an infrared sensor, a sound sensor, or an ultrasound sensor.

7. The detection system of claim 1, wherein at least one sensor within the portable device is mounted on a gimbal to stabilize the sensor.

8. The detection system of claim 1, wherein the portable device includes a first camera mounted on a gimbal and a second camera mounted so as to be rotatable.

9. The detection system of claim 1, wherein the portable device includes an array of cameras and/or sensors arranged in a spiral configuration.

10. The detection system of claim 1, wherein the portable device includes a motor to selectively move one or more of the sensors within the housing.

11. The detection system of claim 1, wherein the device includes active sensors employed in sender/receiver pairs and the sensors are mounted on a pole configured to be rotated by a drive motor.

12. The detection system of claim 1, wherein the body data comprises an image, an infrared image, a sound image, an ultrasonic sound image, or a visual image.

13. The detection system of claim 1, wherein the medical condition or the physiological reaction is associated with an internal organ, an internal system, a blood vessel, or a nerve, heart rate, blood pressure, pupil diameter, an emotional condition, a facial expression, tearing up, swaying, or change in position.

14. The detection system of claim 1, wherein the first type of stimulus is operable to treat the medical condition or change the physiological reaction.

15. The detection system of claim 1, wherein the medical condition comprises one or more of Alzheimer's disease, anemia, a tumor, a brain tumor, a fibroid, diabetic retinopathy, glaucoma, a retinal vascular occlusion, fluid in the lungs, a parasitic worm, an enlarged heart, a heart shape abnormality, pregnancy, a broken bone, bone healing progress, an ovarian cyst, or a change in blood vessel diameter.

16. The detection system of claim 1, wherein the portable device includes a gyro and an accelerometer to determine one or more orientation parameters of the portable device.

17. The detection system of claim 1, wherein the portable device is configured to paired with a second portable device.

18. The detection system of claim 1, wherein the trainable artificial intelligence system utilizes a deep learning neural network, an image and anomaly database, and a medical and image history record of the animal body.

19. A detection system including a portable device configured for detecting physiological or emotional reactions of a subject, comprising:
a housing shaped as a musical instrument;
multiple sensors within the portable device for obtaining body data associated with a physiological reaction or an emotional reaction in the subject, wherein the multiple sensors include first and second sensors, wherein the first sensor is operable to obtain a first type of body data, wherein the second sensor is operable to obtain a second type of body data, and wherein the first and second types of body data are different, and wherein the first sensor or the second sensor comprises a camera;
an audio speaker with a volume control;
a processing tool including a trainable artificial intelligence system that analyses the body data about the subject to identify a physiological or emotional response in the subject in reaction to sounds that the portable device plays; and
a display affiliated with the device to display data from the artificial intelligence system.

20. A method for identifying a medical condition or physiological reaction in an animal body, comprising:
emitting multiple forms of radiation toward an animal body;
obtaining, in response to the multiple forms of radiation, body data representative of the animal body;
providing the body data to a processing tool to identify the presence of one or more medical conditions or physiological reactions of the animal body, wherein the processing tool comprises a trainable AI system; and
providing information concerning the medical condition or physiological reaction to a user interface.
